(19) United States
(12) Patent Application Publication
(10) Pub. No.: US 2009/0312764 A1
(43) Pub. Date: Dec. 17, 2009

(54) INTRAOSSEOUS TRANSPEDICULAR METHODS AND DEVICES

(76) Inventor: James F. Marino, La Jolla, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/137,425

(22) Filed: Jun. 11, 2008

(51) Int. Cl. A61F 5/00; A61B 19/00

(52) U.S. Cl. 606/87; 128/898; 606/86 R

(57) ABSTRACT

This disclosure is directed to minimally-invasive devices, methods and systems for treating vertebral diseases and injuries using multiple therapeutic procedures through small access portals of sufficient dimension that minimize trauma to the patient. More particularly, disclosed herein are devices, methods and systems for an intraosseous transpedicular surgical approach that can be used for a variety of surgical spine procedures.

This disclosure is directed to devices and methods for treating vertebral diseases and injuries. More particularly, disclosed herein are devices and methods for an intraosseous transpedicular surgical approach for a variety of interventions including intervertebral fixation and disc excision/ablation.

### BACKGROUND

A significant number of adults have had an episode of back pain or chronic back pain emanating from a region of the spinal column or backbone. Many people suffering chronic back pain or an injury requiring immediate intervention resort to surgical intervention to alleviate their pain. A number of spinal disorders are caused by traumatic spinal injuries, disease processes, aging processes, and congenital abnormalities that cause pain, reduce the flexibility of the spine, decrease the load bearing capability of the spine, shorten the length of the spine, and/or distort the normal curvature of the spine.

Disc degeneration can contribute to back pain. With age, the nucleus pulposus of the intervertebral discs tends to become less fluid and more viscous. Dehydration of the intervertebral disc and other degenerative effects can cause severe pain in many instances. Annular fissures also may be associated with a herniation or rupture of the annulus causing the nucleus to bulge outward or extrude out through the fissure and impinge upon the spinal column or nerves (a “ruptured” or “slipped” disc).

In addition to spinal deformities that occur over several motion segments, spondylolisthesis (forward displacement of one vertebra over another, usually in the lumbar or cervical spine) is associated with significant axial and/or radicular pain. Anterior column distortion is often accompanied by or caused by a fracture or partial collapse of one or more vertebrae (usually resulting from osteoporosis or traumatic injury) and/or degeneration of a disc. Patients who suffer from such conditions can experience diminished ability to bear loads, loss of mobility, extreme and debilitating pain, and oftentimes suffer neurological deficit in nerve function.

Traditional, conservative methods of treatment include bed rest, pain and muscle relaxant medication, physical therapy or steroid injection. Failure of conservative therapies to treat spinal pain often lead to spinal surgical intervention, with or without instrumentation. Fusion of the vertebrae above and below the degenerate intervertebral disc form a single, solid bone.

Many surgical techniques, instruments and spinal disc implants have been described that are intended to provide less invasive, percutaneous, or minimally invasive access to a degenerated intervertebral spinal disc. Instruments are introduced through the annulus for performing a discectomy and implanting bone growth materials or biomaterials or spinal disc implants within the annulus. One or more annular incisions are made into the disc to receive spinal disc implants or bone growth material to promote fusion, or to receive a pre-formed, artificial, functional disc replacement implant.

Extensive perineural dissection and bone preparation can be necessary for some of these techniques. In addition, the disruption of annular or periannular structures can result in loss of stability or nerve injury. As a result, the spinal column can be further weakened and/or result in surgery-induced pain syndromes.

One technique for spinal fixation includes the immobilization of the spine by the use of spine rods of various configurations that run generally parallel to the long axis of the spine. Typically, the posterior surface of the spine is isolated and bone screws are first fastened to the pedicles of the appropriate vertebrae or to the sacrum and act as anchor points for the spine rods. The bone screws are generally placed two per vertebra, one at each pedicle on either side of the spinous process.

### SUMMARY

There remains a need for minimally-invasive methods, devices and systems for performing multiple therapeutic procedures in the spine through small access portals of sufficient dimension that minimize trauma to the patient.

In one embodiment, disclosed is a method for treating a spinal structure that includes creating an intraosseous channel through a pedicle of a first vertebra, wherein the intraosseous channel extends along an axis of the pedicle from a generally posterior or posterior-inferior aspect to a generally anterior or anterior-superior aspect of the pedicle of the first vertebra. Through at least a portion of the intraosseous channel a spinal region generally superior to the first vertebra and generally inferior to a second vertebra is accessed. The spinal regions can include, for example, the neuroforamina, lateral recess, epidural space, or intervertebral disc space. A treatment device is placed through at least a portion of the channel into or adjacent the spinal region and therapeutic interventions performed on the spinal region. In an embodiment, a surgical guide is used to create the channel and another guide is used to access the spinal region through at least a portion of the channel created. In an embodiment, the therapeutic intervention can be intervertebral distraction. In an embodiment, the therapeutic intervention can include resecting, shaving, shearing, cutting or removing intervertebral disc material. In an embodiment, the therapeutic intervention can be delivering material into the spinal region or a space adjacent thereto, the material including bone growth materials, osteoconductive, osteoinductive, chondroproliferative, chondroreparative, growth factors, osteoproliferative materials, osteogenic proteins, osteoprogenic factor 1, bone morphogenetic proteins (BMP), BMP2 and BMP7. In an embodiment, the vertebrae can be fixed by placing a fixation device such as a pedicle screw through at least a portion of the channel.

### BRIEF DESCRIPTION OF THE DRAWINGS

FIG. 1A is a simplified sagittal view of a vertebrae pair;

FIG. 1B is a simplified, sectional coronal view a vertebrae;

FIG. 2A is a simplified coronal view of the vertebrae pair including a guide pin and support sleeve, the guide pin being inserted into a pedicle according to various embodiments;

FIG. 2B is a simplified sagittal view of the vertebrae pair including a guide pin and support sleeve, the guide pin being inserted into a pedicle as shown in FIG. 2A;

FIG. 2C is a simplified posterior view of the vertebrae pair including a guide pin and support sleeve, the guide pin being inserted into a pedicle as shown in FIG. 2A;

FIG. 2D is a simplified isometric view of the vertebrae pair including a guide pin and support sleeve, the guide pin being inserted into a pedicle as shown in FIG. 2A;

FIG. 3A is a simplified isometric view of the vertebrae pair shown in FIG. 2D further including an obturator and cannula inserted over the guide pin and support sleeve, the obturator being advanced toward a pedicle to create a tissue pathway to the pedicle according to various embodiments;

FIG. 3B is a simplified isometric view of the vertebrae pair where the obturator and guide sleeve have been removed leaving the guide pin inserted into the pedicle with the cannula over the guide pin according to various embodiments;

FIG. 3C is a simplified isometric view of the vertebrae pair shown in FIG. 3B further including a cannulated reamer inserted over the guide pin and within the cannula, the reamer being operatively advanced into the pedicle to form a bore in the pedicle according to various embodiments;

FIG. 4A is a simplified isometric view of the vertebrae pair where the cannulated reamer and the cannula have been removed leaving the guide pin inserted in the bored pedicle according to various embodiments;

FIG. 4B is a simplified isometric view of the vertebrae pair shown in FIG. 4A further including a cannulated spot facer inserted over the guide pin, the spot facer being operatively advanced into the pedicle to enlarge the bore formed in the pedicle according to various embodiments;

FIG. 4C is a simplified isometric view of the vertebrae pair where the cannulated spot facer has been removed leaving the guide pin inserted in the enlarged, bored pedicle according to various embodiments;

FIG. 4D is a simplified isometric view of the vertebrae pair shown in FIG. 4C further including a slotted cannula inserted over the guide pin, the cannula being advanced into the pedicle bore in the pedicle according to various embodiments;

FIG. 5A is a simplified sagittal view of the vertebrae pair shown in FIG. 4D further including a transpedicular channel alignment tool inserted over the cannula according to various embodiments;

FIG. 5B is a simplified sagittal view of the vertebrae pair where the guide pin has been removed leaving the cannula inserted in pedicle bore and transpedicular channel alignment tool inserted over the cannula according to various embodiments;

FIG. 5C is a simplified sagittal view of the vertebrae pair shown in FIG. 5B further including a guide pin with support sleeve, the guide pin and support sleeve being inserted through the transpedicular channel alignment tool's offset guide port and the guide pin advanced at an angle offset from normal to the vertebrae pair into disc space according to various embodiments;

FIG. 5D is a simplified sagittal view of the vertebrae pair shown in FIG. 5C where the cannula in the transpedicular channel alignment tool's normal guide port has been removed leaving the guide pin and support sleeve inserted through the transpedicular channel alignment tool's offset guide port and the guide pin advanced at the offset angle according to various embodiments;

FIG. 6A is a simplified sagittal view of the vertebrae pair shown in FIG. 5D where the support sleeve in the transpedicular channel alignment tool's offset guide port and the alignment tool have been removed leaving the guide pin inserted through a transpedicular channel to the disc space according to various embodiments;

FIG. 6B is a simplified sagittal view of the vertebrae pair shown in FIG. 6A further including a cannulated reamer within a cannula inserted over the offset guide pin, the reamer being operatively advanced into disc space via the transpedicular channel to enlarge the channel according to various embodiments;

FIG. 6C is a simplified sagittal view of the vertebrae pair shown in FIG. 6B where the cannulated reamer and the cannula have been removed leaving the guide pin inserted through the enlarged transpedicular channel to the disc space according to various embodiments;

FIG. 6D is a simplified sagittal view of the vertebrae pair shown in FIG. 6C further including a cannula inserted over the offset guide pin, the cannula being advanced into disc space via the transpedicular channel according to various embodiments;

FIG. 6E is a simplified sagittal view of the vertebrae pair shown in FIG. 6B where the guide pin has been removed leaving the offset cannula in enlarged transpedicular channel to the disc space according to various embodiments;

FIG. 6F is a simplified sagittal view of the vertebrae pair shown in FIG. 6B where bone granules have been inserted into the disc space via the cannula according to various embodiments;

FIGS. 6G and 6I depict the force vectors as applied to a compacted granular-powered material within a cannula and a disc space according to various embodiments.

FIG. 7A is a simplified isometric view of the vertebrae pair shown in FIG. 4D further including a guide pin with support sleeve and second transpedicular channel alignment tool, the guide pin and support sleeve being inserted through the second transpedicular channel alignment tool's offset guide port and the guide pin advanced at a second offset angle from normal to the vertebrae pair into disc space according to various embodiments;

FIG. 7B is a simplified sagittal view of the vertebrae pair shown in FIG. 7A showing that the second offset angle from normal can be greater than the offset angle show in FIGS. 5A to 5D according to various embodiments;

FIG. 7C is a simplified sagittal view of the vertebrae pair shown in FIG. 7B where the cannula in the second transpedicular channel alignment tool's normal guide port has been removed leaving the guide pin and support sleeve inserted through the transpedicular channel alignment tool's offset guide port and the guide pin advanced at the second offset angle according to various embodiments;

FIG. 7D is a simplified sagittal view of the vertebrae pair shown in FIG. 7C where the support sleeve in the second transpedicular channel alignment tool's offset guide port and the second alignment tool have been removed leaving the guide pin inserted through a transpedicular channel to the disc space according to various embodiments;

FIG. 7E is a simplified sagittal view of the vertebrae pair having a cannulated compression-distraction screw advanced over the offset guide pin or wire through the disc space into the superior vertebra via a second transpedicular channel according to various embodiments;

FIG. 7F is a simplified sagittal view of the vertebrae pair having a fusion construct advanced through the inferior vertebra and disc space into the superior vertebra via the second transpedicular channel according to various embodiments;

FIG. 8A is a simplified isometric view of the vertebrae pair shown in FIG. 6D including a reverse pedicle alignment tool inserted over the offset cannula according to various embodiments;

FIG. 8B is a simplified isometric view of the vertebrae pair shown in FIG. 8A including the guide pin inserted in the reverse alignment tool's normal port and through the cannula' slot according to various embodiments;

FIG. 8C is a simplified isometric view of the vertebrae pair shown in FIG. 8B where the cannula has been removed and the guide pin has been advanced into the pedicle normal channel according to various embodiments;

FIG. 8D is a simplified isometric view of the vertebrae pair shown in FIG. 8C where the reverse pedicle alignment tool has been removed according to various embodiments;

FIG. 8E is a simplified sagittal view of the vertebrae pair shown in FIG. 8D where the guide pin is inserted into the normal pedicle channel according to various embodiments;

FIG. 8F is a simplified coronal view of the vertebrae pair shown in FIG. 8E where the guide pin is inserted into the normal pedicle channel according to various embodiments.

FIGS. 9A to 9F are diagrams of a transpedicular channel alignment and access tool according to various embodiments.

FIGS. 10A to 10F are diagrams of the transpedicular channel alignment and access tool employed in a vertebral pedicle according to various embodiments.

### DETAILED DESCRIPTION

Disclosed are methods and devices for accessing and treating the spine, while minimizing trauma to surrounding tissue. The present disclosure relates generally to spinal surgery, particularly methods and apparatus for forming one or more intraosseous access bores in a minimally invasive, low trauma, manner and providing a therapy to the spine employing the common intraosseous bore.

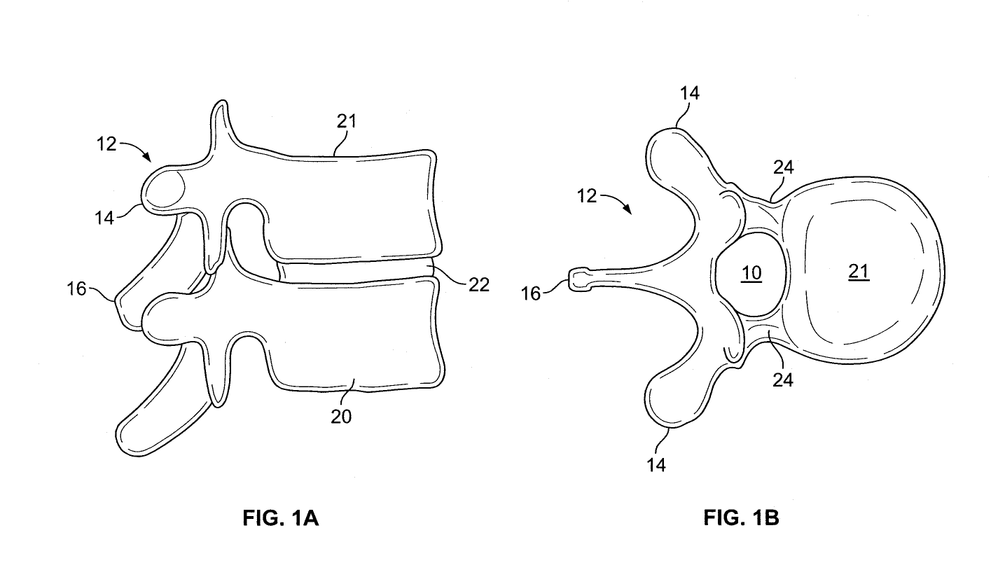

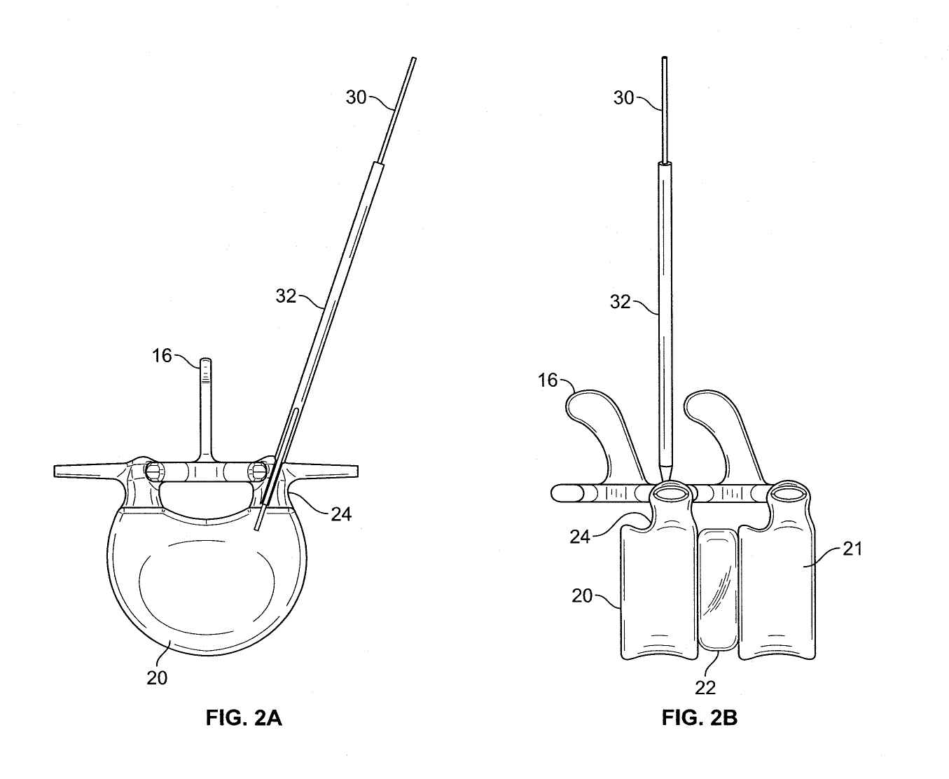

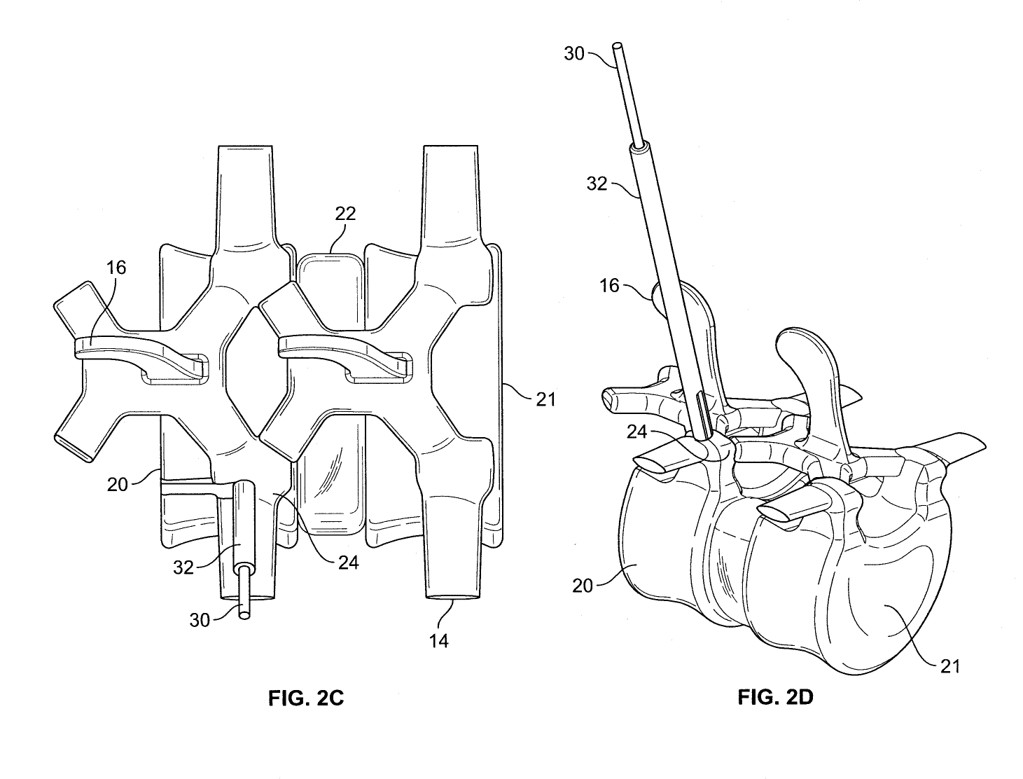

FIG. 1A is a simplified sagittal view of a vertebrae pair 20, 21. FIG. 1B is a simplified, sectional coronal view of the vertebrae 21 of the vertebrae pair shown in FIG. 1A. Each vertebra 20, 21 includes lamina 12, transverse processes 14, a spinous process 16, central canal 10, and pedicles 24. A disc 22 comprised of an annulus and disc nucleus (not shown) is located between the vertebrae pair 20, 21. Due to disc degeneration, expulsion, annulus tears, or other conditions, the spinal cord that passes through the central canal 10 can become compressed causing patient discomfort. It can be desirable to modify or fix the spatial relationship between the vertebrae pair 20, 21. FIGS. 2A to 8F present various apparatus and methods for accessing the vertebrae pair 20, 21 to perform a surgical procedure.

It can be desirable to access the disc space or superior vertebra in order to decompress nerves by removing herniated or prolapsed discs. Ablation and/or excision techniques can be performed using a transosseous, transpedicular approach as described herein. In an embodiment, access to the disc space 22 or superior vertebra 21 can be achieved via a channel formed in an inferior vertebra pedicle 24, such as one immediately inferior to the disc space or vertebra to be entered. FIGS. 2A to 7D present methods and apparatus for forming such a channel according to various embodiments. In this embodiment a normal channel can be formed in the inferior vertebrae pedicle. Through the formed normal channel an offset channel can be created. The offset channel based on the formed normal channel can enable access, for example to the disc space, superior vertebrae 21, vertebral endplate, neuroforamina, epidural space, lateral recess or the like. The offset channel can be used for a number of procedures such as disc resection, excision, endplate decortication, vertebral reduction or compression, delivery of material etc. The normal channel can also be used for a number of procedures such as subsequent pedicle screw fixation, for example. Methods described herein use a common entry for a variety of procedures, for example intervertebral fixation of the inferior vertebra as well as excision, ablation resection, shaving, shearing, cutting or removing of the intervertebral disc material or vertebral reduction or compression.

The path used can be located on or about the accessory process of the inferior vertebra of a motion segment (on the posterior margin of the vertebra, just lateral to the superior articular process, immediately superior to the pars interarticularis, at the root of the transverse process, and immediately posterior to the pedicle). A guide can be employed to place a pin through the posterior or posterior inferior aspect of the pedicle from the caudal to the cephalad direction, or generally along the axis of the pedicle from a generally posterior or posterior-inferior aspect to a generally anterior or anterior-superior aspect of the pedicle on an inferior pedicle. The pin can enter the foramen or the juncture of the foramen and the posterolateral annulus (within or adjacent to the lateral recess of the spinal canal). The method of creating this path is applicable to all thoracic and lumbar levels and can be used in the cervical spine with some modification.

FIG. 2A is a simplified coronal view, FIG. 2B is a simplified sagittal view, FIG. 2C is a simplified posterior view, and FIG. 2D is an isometric view of the vertebrae pair 20, 21 including a guide pin or wire 30 and support sleeve 32 according to various embodiments. In one embodiment, the guide pin 30 can be inserted at a posterior, lateral angle from the coronal view and normal to the vertebrae 20 from the sagittal view. The guide pin extends into the vertebrae 20 pedicle 24 while not violating the pedicle wall. In addition in an embodiment a support sleeve 32 can be inserted over the guide pin 30. The support sleeve 32 can be a thin walled cannula in an embodiment of the device. In an embodiment, the most posterior cross-sectional area of the intraosseous channel in the pedicle overlaps, is contiguous or confluent with at least a portion of the most posterior aspect of the guide pin 30 within the pedicle.

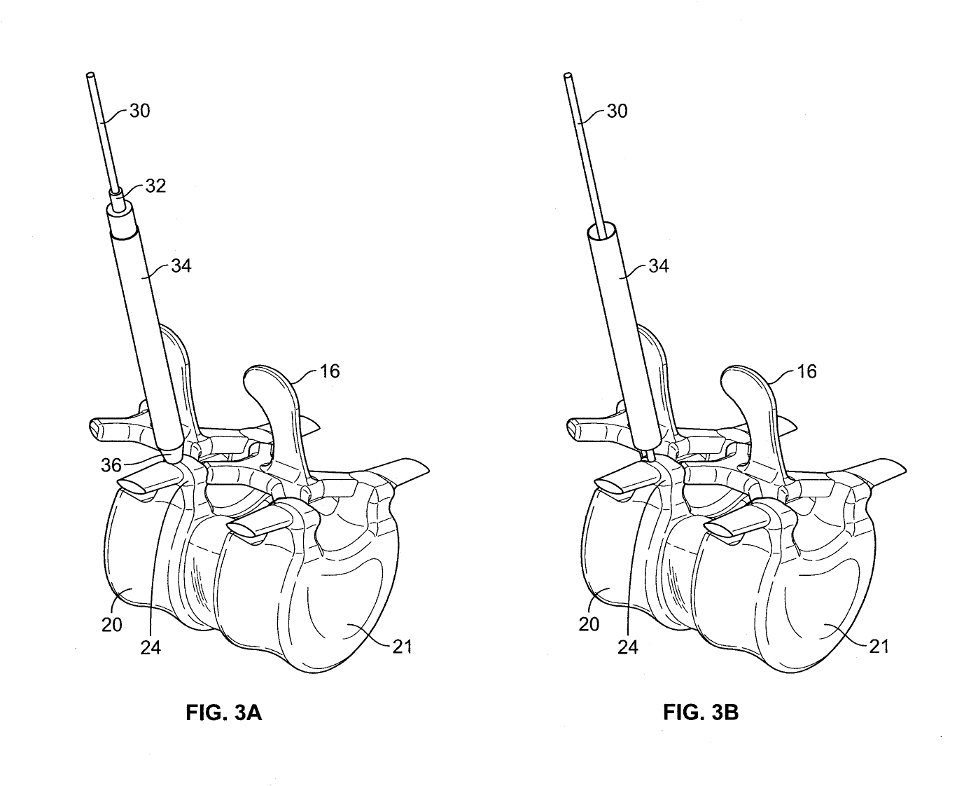

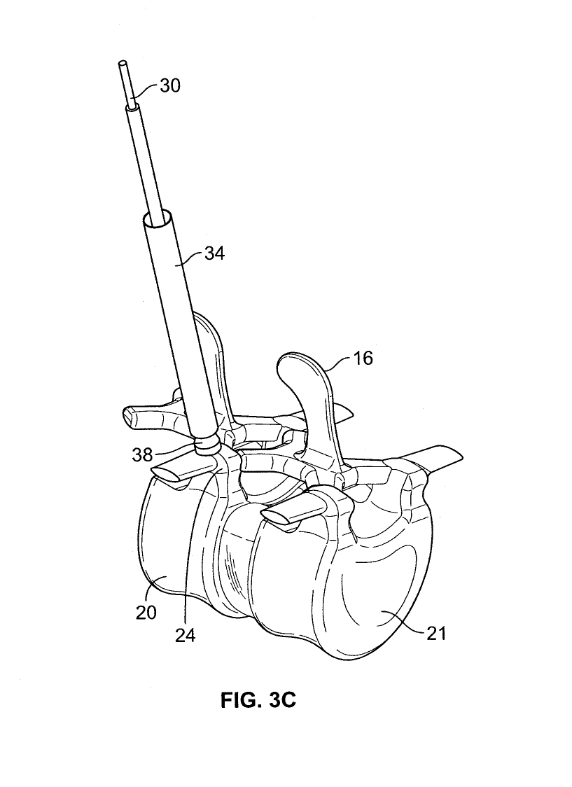

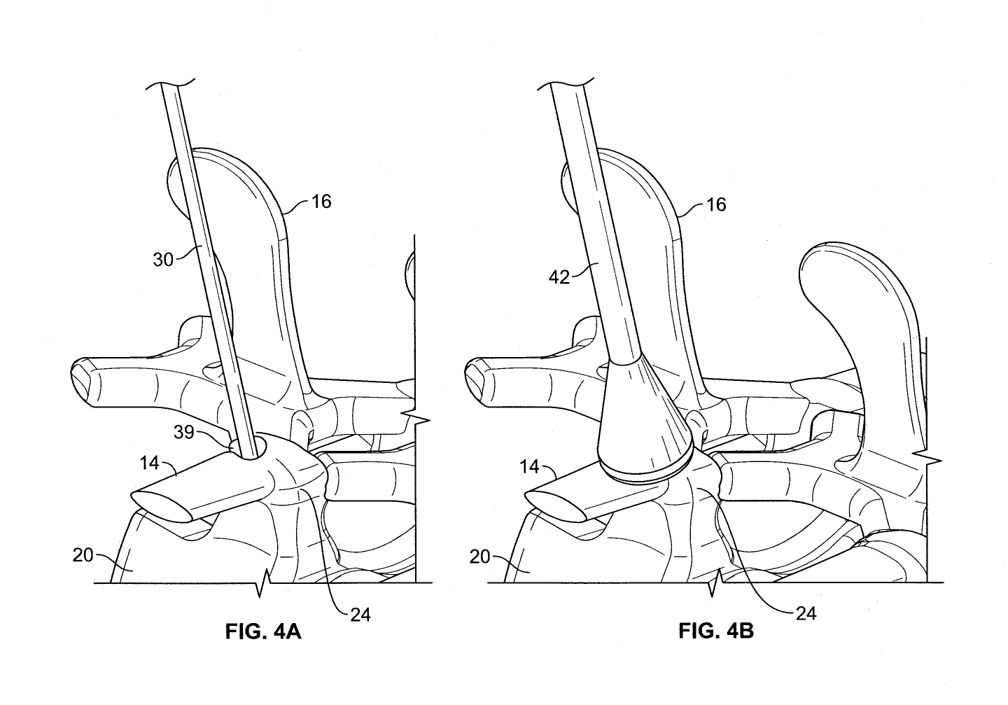

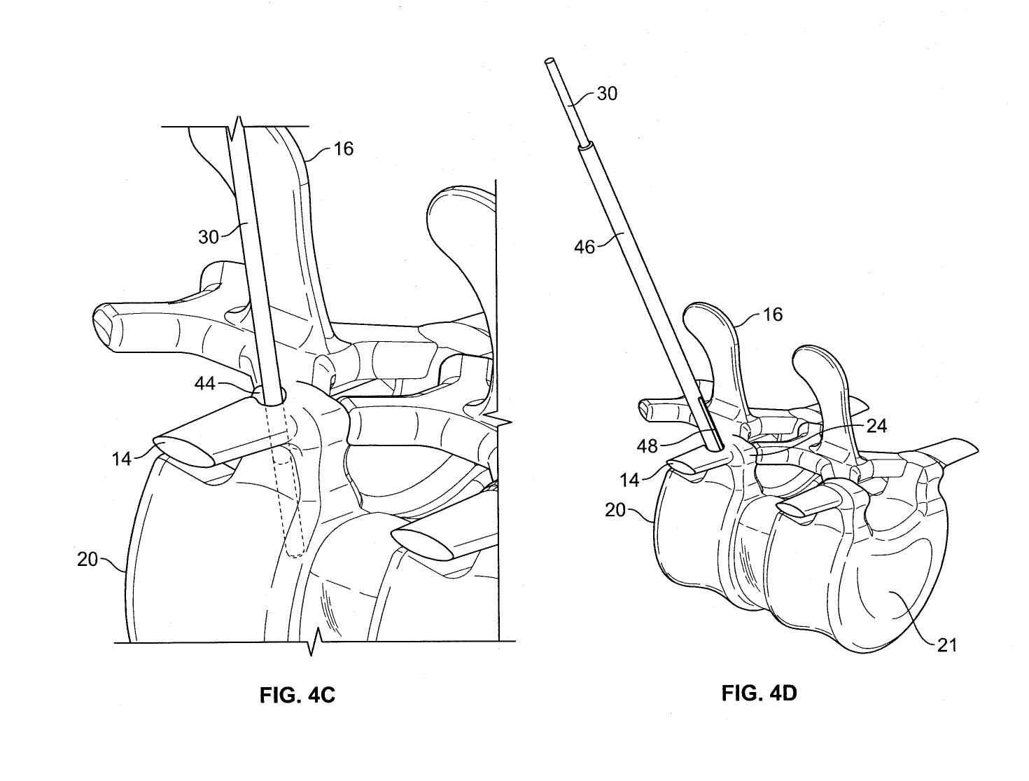

FIG. 3A is a simplified isometric view of the vertebrae pair 20, 21 shown in FIG. 2D further including an obturator 36 and cannula 34 inserted over the guide pin 30 and support sleeve 32. In an embodiment the obturator 36 can be advanced toward a pedicle 24 to create a tissue pathway to the pedicle 24. FIG. 3B is a simplified isometric view of the vertebrae pair 20, 21 where the obturator 36 and guide sleeve 32 have been removed leaving the guide pin 30 inserted into the pedicle with the cannula 34 over the guide pin 30. FIG. 3C is a simplified isometric view of the vertebrae pair 20, 21 shown in FIG. 3B further including a cannulated reamer 38 inserted over the guide pin 30 and within the cannula 34. In an embodiment, the reamer 38 can be operatively advanced into the pedicle 24 to form a bore in the pedicle 24. In an embodiment the reamer 38 can have about a 5 mm diameter and about an 8 mm depth stop. In this embodiment, the reamer 38 can be used to form an approximately 10 mm deep, 5 mm in diameter bore (39 shown in FIG. 4A) in the pedicle 24, the bore 39 axis being approximately normal to the coronal plane of vertebrae 20. In this embodiment the cannula 34 can have a diameter of about 8.5 mm.

FIG. 4A is a simplified isometric view of the vertebrae pair 20, 21 where the cannulated reamer 38 and the cannula 34 have been removed leaving the guide pin 30 inserted in the bored pedicle according to various embodiments. FIG. 4B is a simplified isometric view of the vertebrae pair 20, 21 shown in FIG. 4A further including a cannulated spot facer 42 inserted over the guide pin 30. In an embodiment, the spot facer 42 can be operatively advanced into the pedicle 24 to enlarge an upper section of the bore 39 formed in the pedicle 24. In an embodiment the spot facer 42 has about a 12 mm diameter with a projected wall. In an embodiment the spot facer 42 forms a larger upper bore section to be occupied by a polyaxial or monoaxial pedicle receiving section, the section moveably coupled or couplable to a pedicle screw head.

FIG. 4C is a simplified isometric view of the vertebrae pair 20, 21 where the cannulated spot facer 42 has been removed leaving the guide pin 30 inserted in the enlarged, bored pedicle according to various embodiments. FIG. 4D is a simplified isometric view of the vertebrae pair 20, 21 shown in FIG. 4C further including a slotted cannula 46 inserted over the guide pin 30, the cannula 46 being advanced into the pedicle bore 44 in the pedicle 24 according to various embodiments.

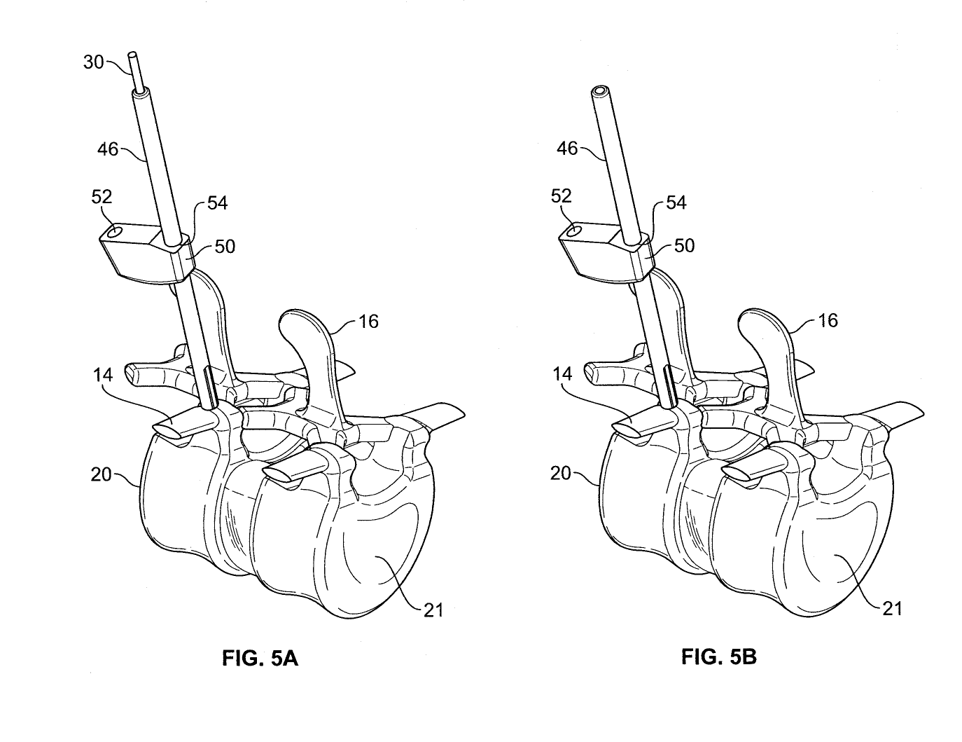

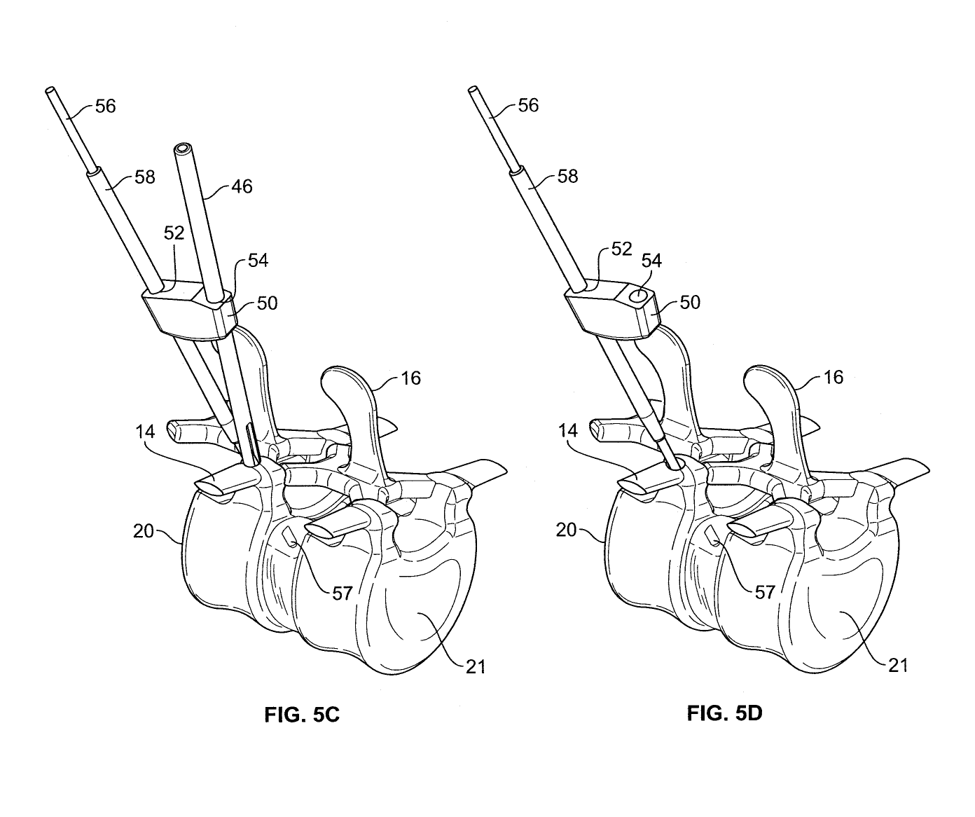

FIG. 5A is a simplified sagittal view of the vertebrae pair 20, 21 shown in FIG. 4D further including a transpedicular channel alignment tool 50 inserted over the cannula according to various embodiments. In an embodiment the alignment tool 50 is aligned along the caudal-cephalad (sagittal) plane. The tool 50 includes a normal port 54 and an offset port 52. The normal port can be sized to receive the guide pin 30 or slotted cannula 46.

FIG. 5B is a simplified sagittal view of the vertebrae pair 20, 21 where the guide pin 30 has been removed leaving the slotted cannula 46 inserted in a pedicle bore 44 and transpedicular channel alignment tool 50 inserted over the cannula 46 according to various embodiments. In one embodiment the normal port 54 of the alignment tool 50 can be sized to receive the slotted cannula 46. In another embodiment the offset port 52 can be oriented at about a 20 degree angle to the normal port 54. FIG. 5C is a simplified sagittal view of the vertebrae pair 20, 21 shown in FIG. 5B further including an offset guide pin 56 with an offset support sleeve 58 inserted through the offset guide port 52 of the transpedicular channel alignment tool 50. In another embodiment the offset guide pin 56 can be advanced at an angle offset from normal to the vertebrae pair 20, 21 into disc space. In another embodiment, one or more X-rays can be taken and reviewed to determine whether the offset guide pin 56 is proceeding along a desired pathway in the pedicle 24 prior to advancement into the disc space 22.

In an alternative embodiment, a surgical guide (such as a redirection guide) can be employed that is anchored or positioned on or through the posterior entrance of the pedicle. The redirection guide can have an adjustable element that directs a surgical path through the vertebra, for example on or near the accessory process along or near to the posterior aspect of the pedicle entrance; and directed from generally posterior to anterior and generally caudal to cephalad. The redirection guide utilizes a bifurcated and stepped diameter sleeve to place a redirection pin in one of various paths that converge on the posterior pedicle access path. The redirection guide does not require preoperative determination of the “bony pathway” and does not require precise placement of a “localizing pin” to a specific depth. In an embodiment, the most posterior cross-sectional area of the intraosseous channel in the pedicle overlaps, is contiguous or confluent with at least a portion of the most posterior aspect of the guide within the pedicle.

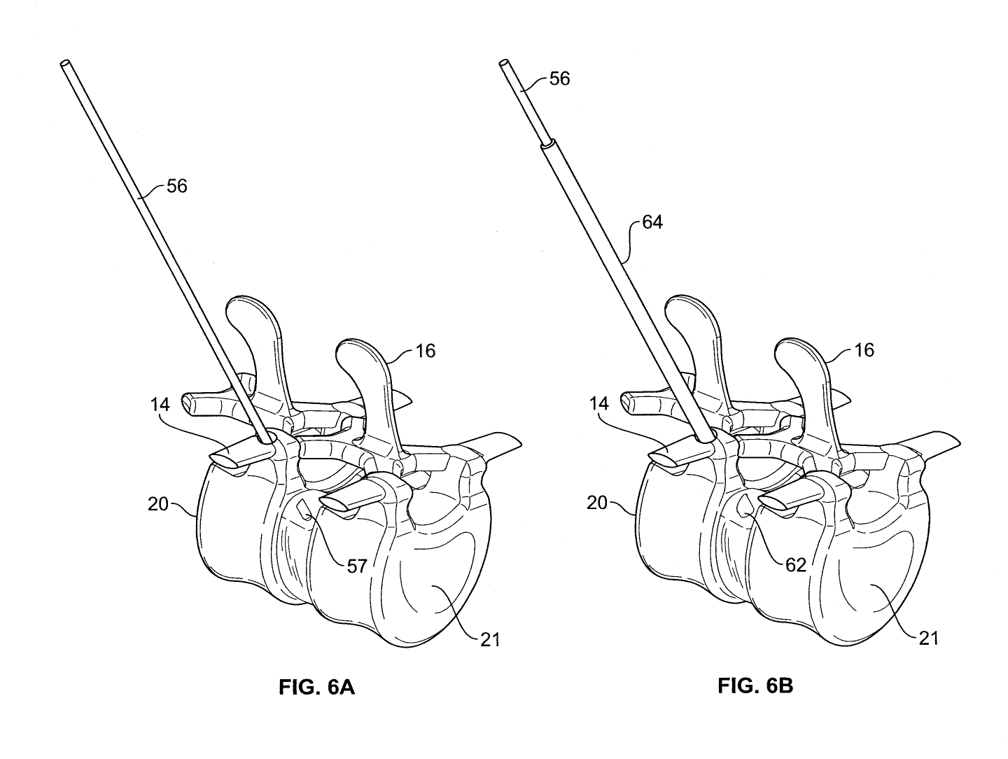

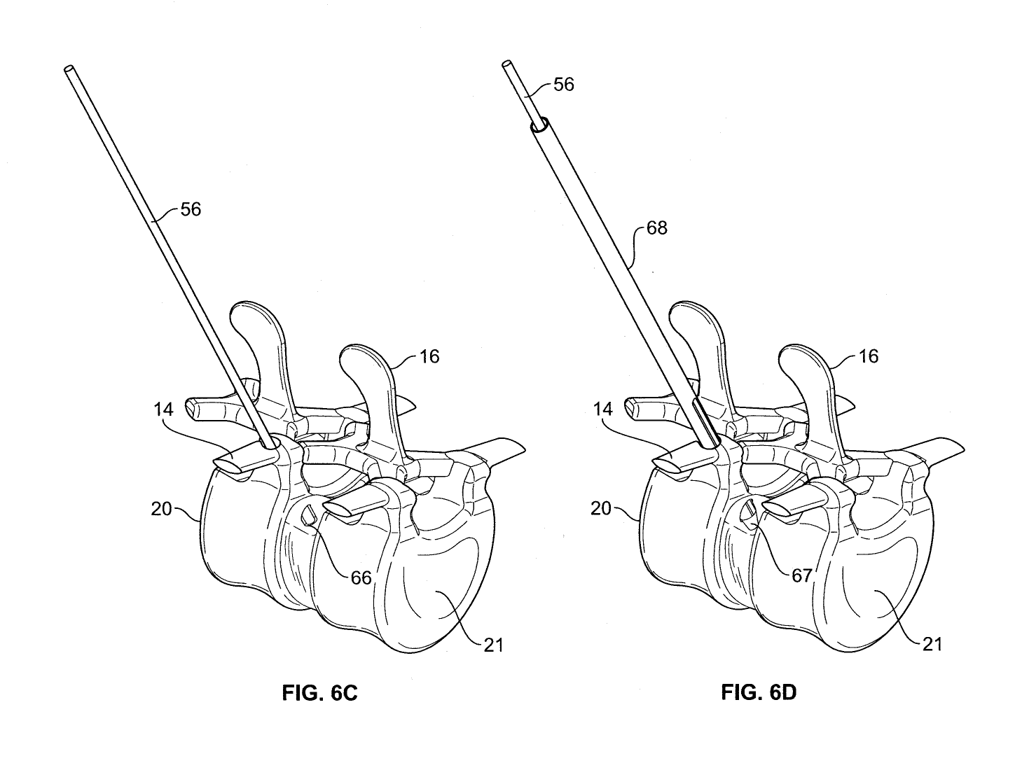

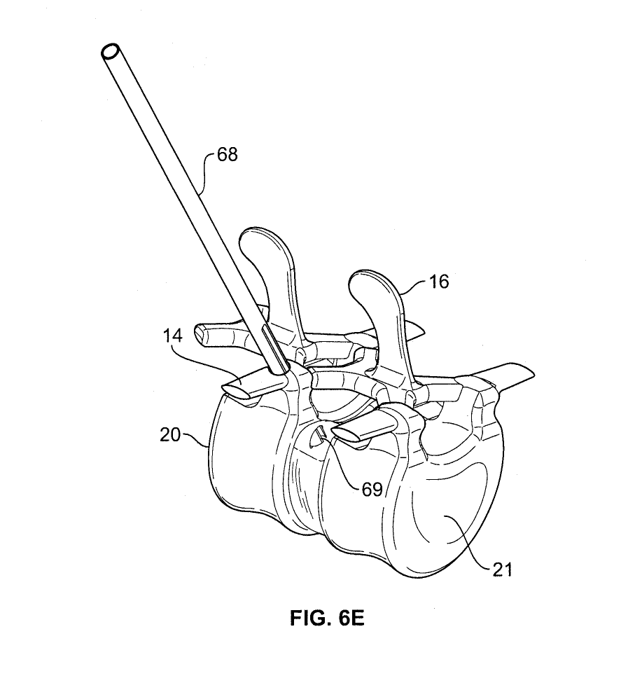

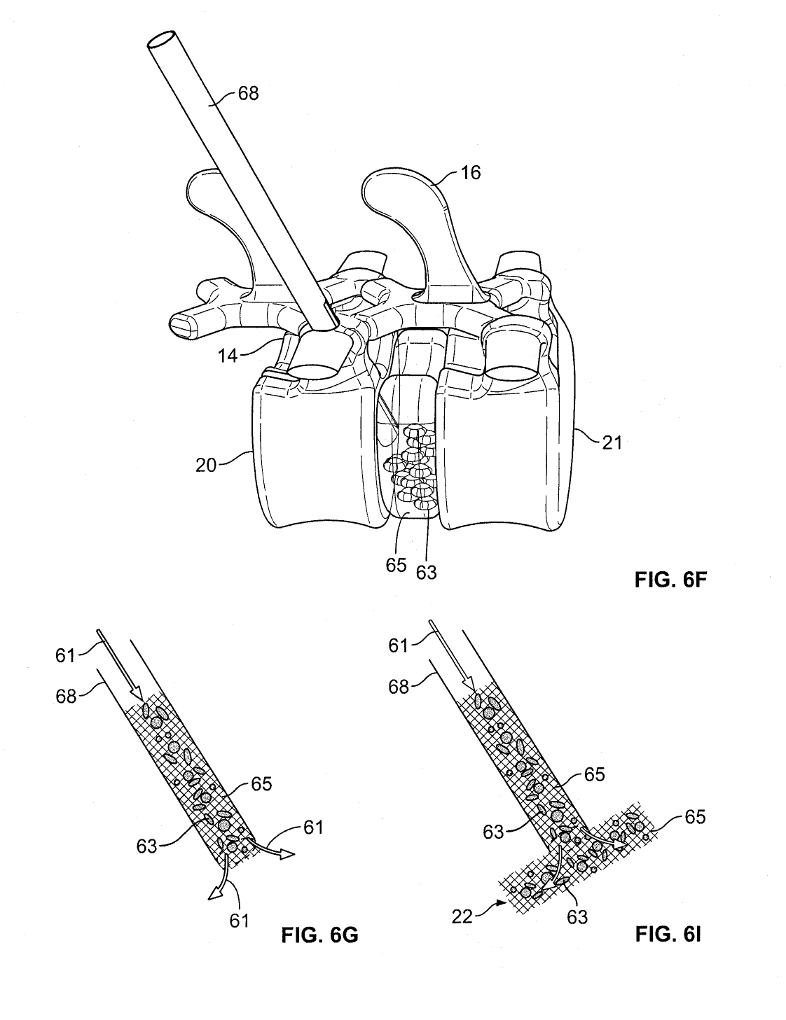

FIG. 5D is a simplified sagittal view of the vertebrae pair 20, 21 shown in FIG. 5C where the cannula 46 in the normal guide port 54 of the transpedicular channel alignment tool has been removed. FIG. 5D shows an offset guide pin 56 and support sleeve 58 inserted through the offset guide port 52 of the transpedicular channel alignment tool 50 and the offset guide pin 56 advanced at the offset angle according to various embodiments. FIG. 6A is a simplified sagittal view of the vertebrae pair 20, 21 shown in FIG. 5D where the offset support sleeve 58 and the alignment tool 50 have been removed leaving the offset guide pin 56 inserted through a transpedicular channel to the disc space 22 according to various embodiments. As shown, the tip 57 of the guide pin 56 can project into the disc space 22. In an embodiment the transpedicular channel can be enlarged to enable different procedures to be performed in the disc space 22. The transpedicular channel can be positioned so that it is not adjacent or near any nerve pathways in one embodiment, reducing the risk of nerve related injuries due to a procedure being performed in the disc space 22.

FIG. 6B is a simplified sagittal view of the vertebrae pair 20, 21 shown in FIG. 6A further including a cannulated reamer 62 within a cannula 64 inserted over the offset guide pin 56. In an embodiment the reamer 62 can be operatively advanced into disc space via the transpedicular channel to enlarge the channel 66. In an embodiment the reamer 62 can be about a 5.5 mm reamer to form a 5.5 mm diameter channel 66 from the pedicle 24 of the inferior vertebra 20 to the disc space 22. FIG. 6C is a simplified sagittal view of the vertebrae pair 20, 21 shown in FIG. 6B where the cannulated reamer 62 and the cannula 64 have been removed leaving the guide pin 56 inserted through the enlarged transpedicular channel 66 to the disc space 22.

FIG. 6D is a simplified sagittal view of the vertebrae pair shown in FIG. 6C further including a slotted cannula 68 and obturator 67 inserted over the offset guide pin 56. In an embodiment a tapered obturator 67 within a slotted, thin walled cannula 68 are inserted over the offset guide pin 56 into the disc space 22 via the transpedicular channel 66. In an embodiment the slotted cannula 68 has about a 5.5 mm diameter to be accommodated by the channel 66 formed by the reamer 62. FIG. 6E is a simplified sagittal view of the vertebrae pair 20,21 shown in FIG. 6D where the guide pin 56 and obturator 67 have been removed leaving the slotted, offset cannula 68 in the enlarged transpedicular channel 66 to the disc space 22 according to various embodiments.

Various tools and instruments can be employed via the cannula 68 to perform procedures such as within the disc space 22 using at least a portion of the intraosseous channel. For example, it might be desirable to use the transosseous transpedicular approach to remove disc material, osteophytes or other structures (e.g. facet capsule or facet joint) that might be impinging on the nerve root(s), including herniated or prolapsed disc material. Other procedures that can be performed through a portion of the intraosseous channel in addition to discectomy, include placement of disc arthroplasty devices, endplate “decortication”, annulus closure or repair, fusion implantation including implants, distraction devices, spacers or cages. Implantation of therapeutic materials such as bone growth materials, nuclear replacement material, and allograft material, and introduction of osteoinductive, osteoconductive or osteoproliferative agents are also considered herein. More specifically, therapeutic bone growth materials such as osteogenic proteins including osteoprogenic factor 1 and bone morphogenetic proteins (BMP) including BMP2 and BMP7.

FIG. 6F is a simplified sagittal view of the vertebrae pair shown in FIG. 6B where bone granules 63 (allograft material) packed with a powdered material 65 have been inserted into the disc space 22 via the cannula 68 according to various embodiments. FIGS. 6G and 6I depict the force vectors 61 as applied to the compacted granular-powdered material 63,65 within a cannula 68 and disc space 22. The bone granules 63 can be packed with a powdered material 65 to facilitate their passage into the disc space 22 via the cannula 68. In particular, the powdered material 65 helps prevent the larger particles 63 from binding together and becoming wedged within a cannula 68 as passed therethrough. As shown in FIG. 6G the force vectors 61 can split at the cannula distal end as the powdered material 65 and granules 63 become disassociated as the cannula 68 walls prevent earlier such disassociation.

In an embodiment the powdered material can be calcium sulfate, Plaster of Paris (calcium sulfate hemi-hydrate), finely pulverized cortical bone with decalcification, or similar fine material safe for insertion into the disc space 22 and possible absorption. In an embodiment the granules 63 can be a granular cortical or structural allograft material. The granules 63 can have a generally spherical geometry and maximum cross sectional area smaller than the cross section area of a delivery cannula 68. In an embodiment a binding agent can be employed to bind the powdered material 65 and granules 65 including evaporated or saturated sugar or starch solution. The granular composite (65 and 63 and binding agent) can be fashioned into cylindrical pellets using a thermal and pressure modulated curing process. The resultant pellets can then be sterilely packaged.

In an embodiment the cylindrical pellets can be packaged within a thin walled polymer material, e.g. “straws”. Such packages (pellets with straws) can be inserted into a delivery cannula 68 or alternatively placed in automated delivery devices or systems. Such cylindrical pellets can be driven via linear forces 61 through the length of the cannula 68, without pellet dissociation or granular element binding. As noted once the composite (63, 65) exits the supportive cannula 68 walls additional forces (e.g. impact loading upon vertebra 20, 21 and disc annulus 22 can dissociate the granules 63. Such dissociation can form an expanding sphere of composite material, the sphere capable of effecting bone displacement or fracture site reduction and having load bearing capacity proportional to the material 63 density. The powdered material 61 granules 65 composition can be used in cannulated procedures for intervertebral disc arthrodesis, vertebroplasty applications for vertebral compression fractures, periarticular depression fracture reductions and bone grafting, bone cyst therapies, etc.

The alignment tool 50 can create an offset angle of, for example, about 20 degrees of normal that can be used to form a transpedicular pathway or channel to a disc space via an inferior vertebra 20. In another embodiment it can be desirable to access the lower endplate of the superior vertebra 21 in addition to the disc space 22. The osseous channel can terminate in the disc space through the posterior aspect of the superior endplate of the vertebra. Alternatively, the osseous channel can terminate along the superior aspect of the pedicle or at the pedicle-vertebral body juncture, entering the neuroforamina at or near the annular ligament attachment site. This surgical pathway can be used for disc or bone resection for the decompression of nerve roots, intervertebral disc excision, endplate “decortication”, insertion of nuclear replacement material, insertion of intervertebral disc arthroplasty devices, introduction of osteoinductive agents, osteoconductive agents, osteoproliferative agents, intervertebral distraction devices, and/or intervertebral spacers or cages.

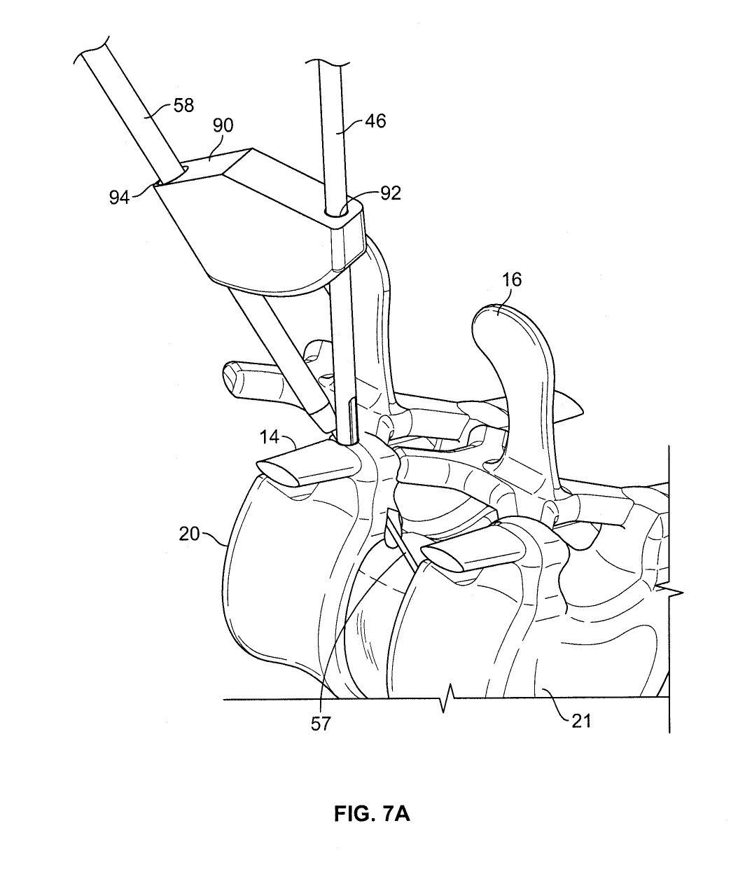

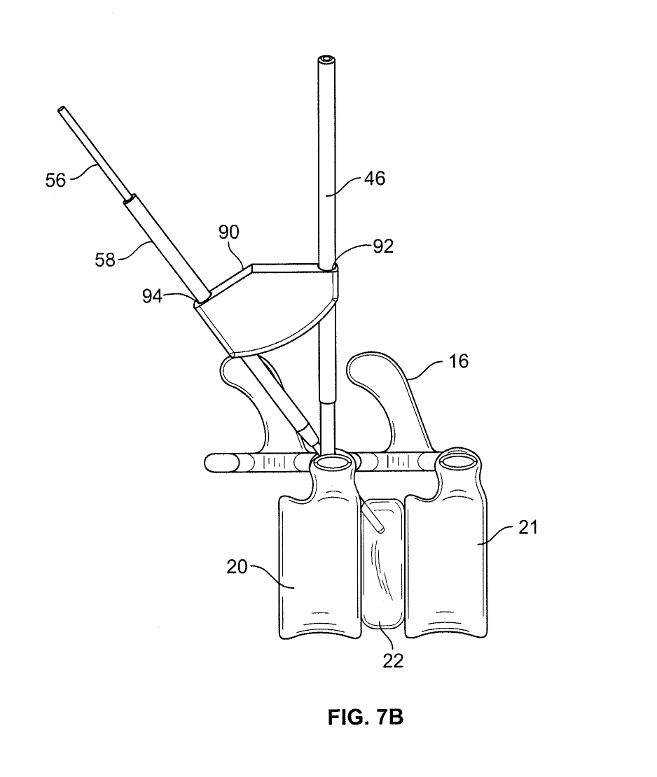

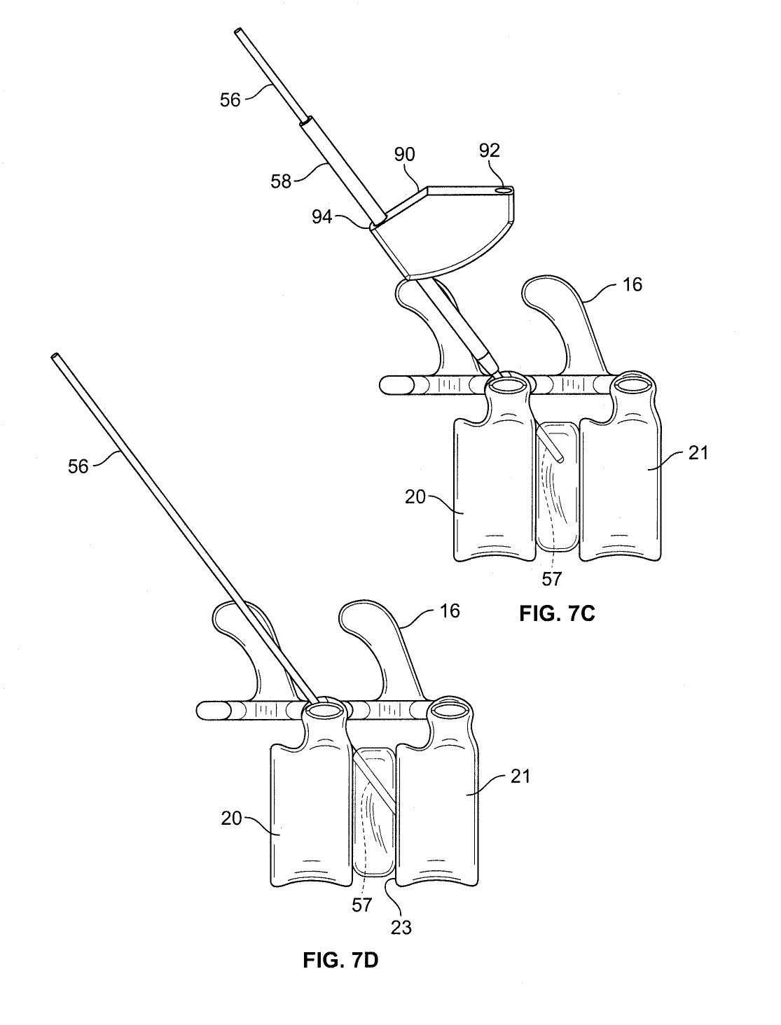

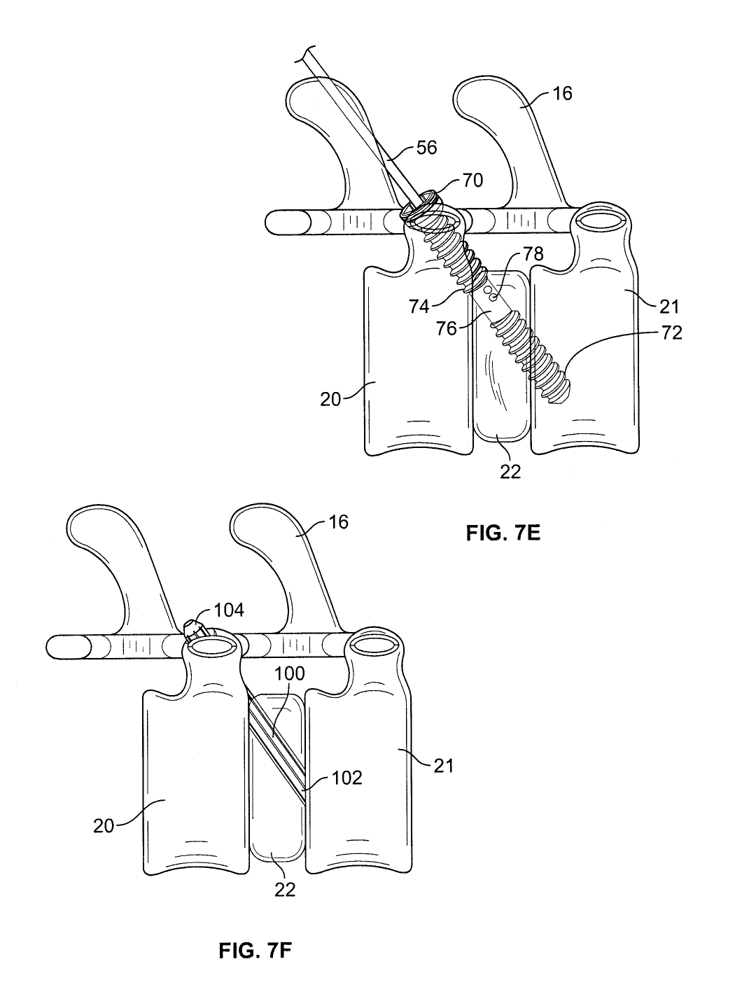

FIG. 7A is a simplified isometric view of the vertebrae pair 20, 21 shown in FIG. 4D further including an offset guide pin 56 with support sleeve 58 and an embodiment of a transpedicular channel alignment tool 90. In this embodiment the alignment tool 90 creates an offset angle of about 35 degrees relative to the normal port 92. In this embodiment the offset guide pin 56 and support sleeve 58 are inserted through the offset guide port 94. The greater offset angle provided by the alignment tool 90 can enable the guide pin to be advanced through the disc space 22 and into the lower endplate 23 of the superior vertebra 21 (see FIG. 7D) according to various embodiments. FIG. 7B is a simplified sagittal view of the vertebrae pair 20, 21 shown in FIG. 7A. FIG. 7B shows an embodiment where a second offset angle from normal greater than the offset angle shown in FIGS. 5A to 5D according to various embodiments.

FIG. 7C is a simplified sagittal view of the vertebrae pair 20, 21 shown in FIG. 7B where the cannula 46 has been removed leaving the offset guide pin 56 and support sleeve 58 inserted through the guide port 94. The offset guide pin 56 tip 57 has been inserted into the disc space 22. FIG. 7D is a simplified sagittal view of the vertebrae pair 20, 21 shown in FIG. 7C where the offset support sleeve 58 and the second alignment tool 90 have been removed leaving the guide pin 56 inserted through a transpedicular channel to the disc space 22 according to various embodiments. As described above in the formation of the transpedicular channel 66, a cannulated reamer 62 within a sleeve can be provided to create an enlarged pathway through the disc space 22 and into the endplate 23. Then a thin walled, slotted cannula 68 and obturator 67 pair can inserted over the guide pin 56 and the guide pin 56 and obturator 67 removed leaving the slotted cannula 68 extending into the endplate 23. It is contemplated herein that these procedures can be performed with or without a redirection guide.

Procedures can be performed within the disc space and into the superior vertebra 21 through the offset transpedicular channel. In an embodiment, accessing the intervertebral disc space involves distracting the superior and inferior vertebrae with a distracter device. For example, FIG. 7E is a simplified sagittal view of the vertebrae pair 20, 21 having a cannulated compression-distraction screw 70 advanced over the offset guide pin or wire 56 through the disc space into the superior vertebra via an offset transpedicular channel according to various embodiments. The compression-distraction screw 70 has distal thread 72, proximal thread 74, non-threaded central section 76, and locking ports 78. In an embodiment, the distal thread 72 can be independently rotated via a head within the central section 76. In an embodiment the distal threaded portion 72 can have a sleeve within the central section 76 so the portion 72 can extend away or toward the portion 74.

In another embodiment other instrumentation can be inserted into the superior vertebra 21 via the transpedicular channel. FIG. 7F is a simplified sagittal view of the vertebrae pair 20, 21 having a fusion construct advanced through the inferior vertebra 20 and disc space 22 into the superior vertebra 23 via the second transpedicular channel according to various embodiments. In this embodiment the construct can be a bone dowel having a proximal 84 and distal end 82. The distal end 82 of the bone dowel 80 can be embedded into the superior vertebra 21 endplate 23 and its proximal end 84 in the inferior vertebra. In an embodiment a portion of the disc 22 can be removed and replaced with implants, bone growth materials, or allograft material prior to the fusion construct 80 insertion/implantation. The transpedicular channel into the superior vertebra 21 can also be used to perform kyphoplasty and other vertebra height restoration and modification procedures.

After performing one ore more procedures via the offset transpedicular channel, it can be desirable to access the normal pedicle channel 44 to perform one or more procedures via the normal pedicle channel 44. For example, a pedicle screw can be inserted through the common intraosseous transpedicular entry for subsequent pedicle screw fixation. In an embodiment, the fixation device implanted can threadably engage one vertebra, such as a vertebra pedicle inferior to a target disc space. In an embodiment, the fixation device implanted can threadably engage at least one or more than one vertebra, such as a vertebra pedicle inferior and a vertebra pedicle superior to a target disc space. A common intraosseous transpedicular entry for both pedicle screw fixation and other spinal procedures such as procedures within the disc space provides advantages, for example better pedicle screw performance and screw purchase.

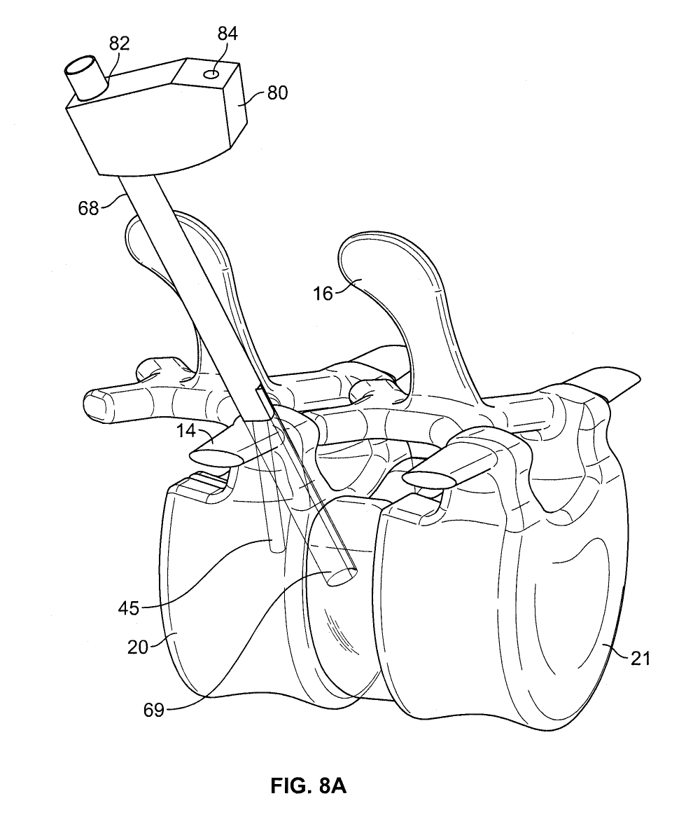

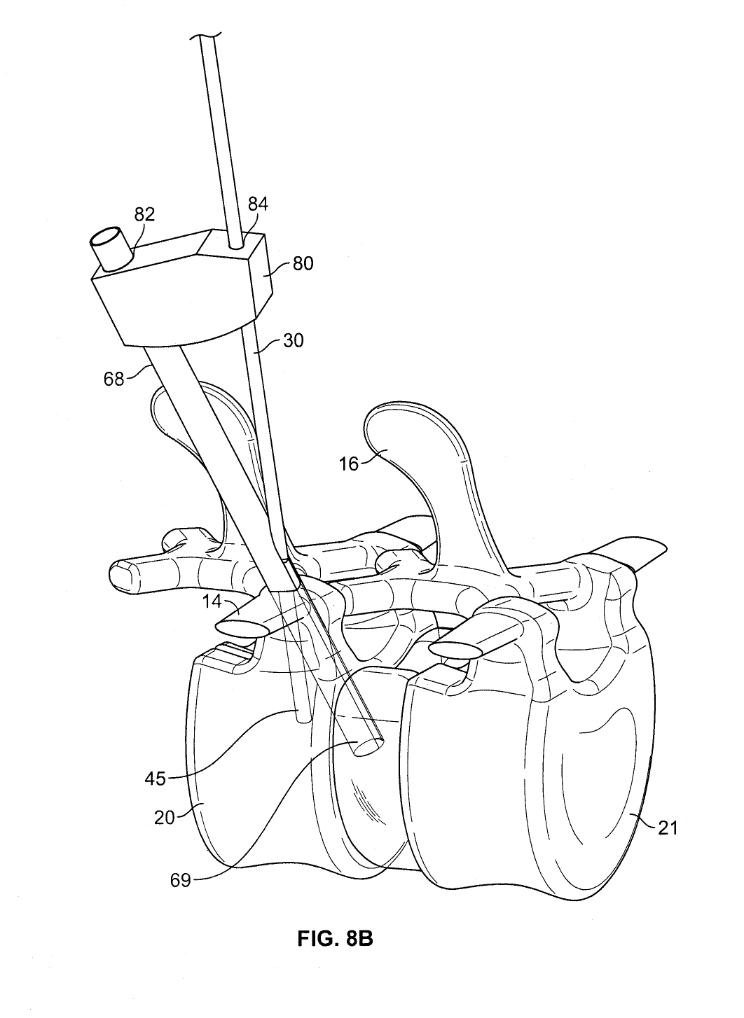

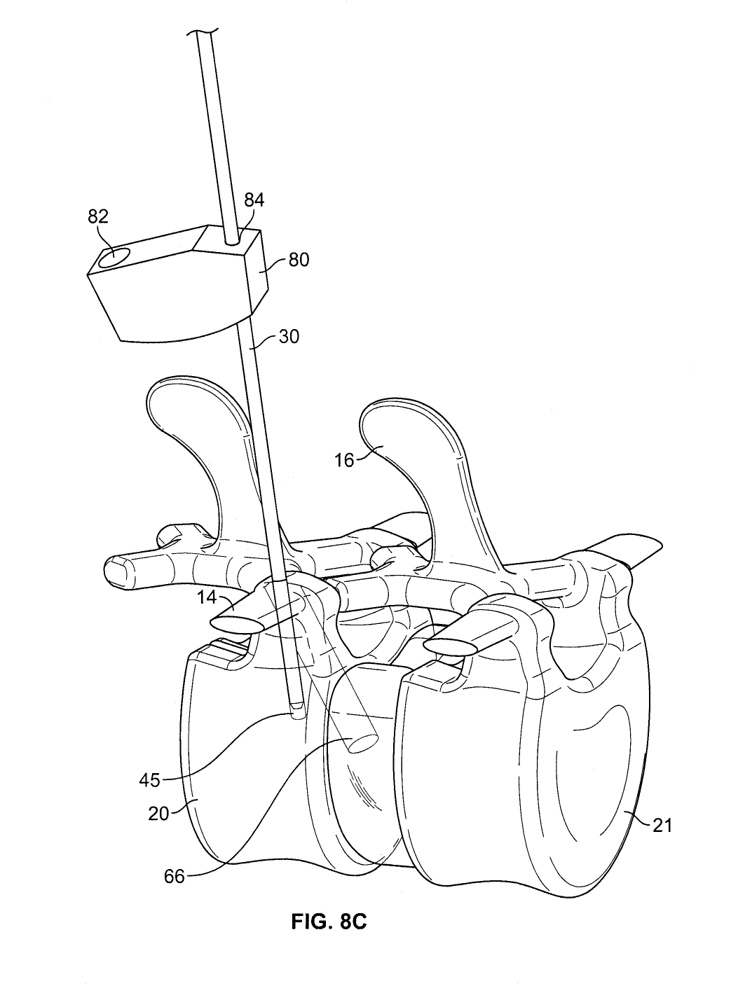

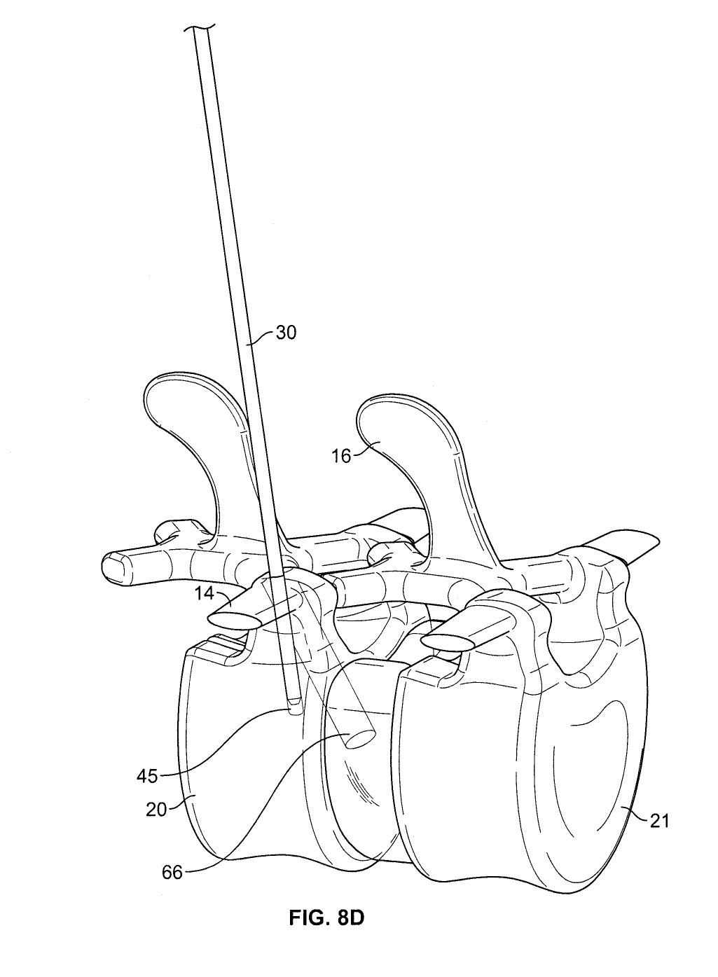

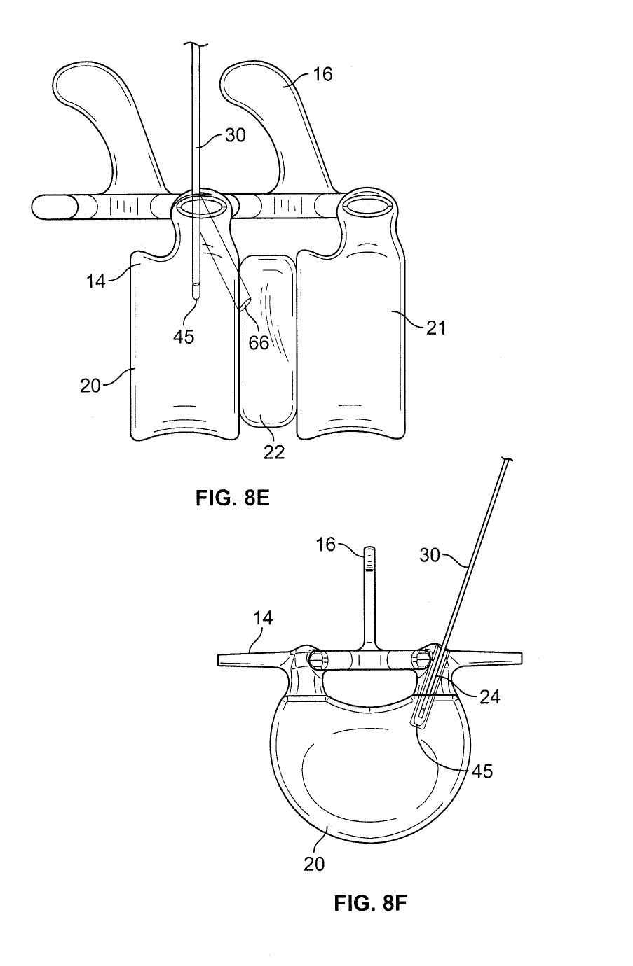

FIG. 8A is a simplified isometric view of the vertebrae pair shown in FIG. 6D including a reverse pedicle alignment tool 80 inserted over the offset cannula 68 according to various embodiments. The reverse pedicle alignment tool 80 includes an offset guide port 82 and a normal guide port 84. The offset guide port 82 can be sized to fit the offset cannula 68. FIG. 8B is a simplified isometric view of the vertebrae pair shown in FIG. 8A including the guide pin 30 inserted in the normal guide port 84 of the reverse alignment tool 80. In an embodiment the guide pin 30 passes through the cannula 68 slot 69.

FIG. 8C is a simplified isometric view of the vertebrae pair 20, 21 shown in FIG. 8B where the offset cannula 68 has been removed and the guide pin 30 has been advanced into the pedicle normal channel 45 according to various embodiments. FIG. 8D is a simplified isometric view, FIG. 8E is a simplified sagittal view, and FIG. 8F is a simplified coronal view of the vertebrae pair 20, 21 shown in FIG. 8C where the reverse pedicle alignment tool 80 has been removed leaving the guide pin 30 in the channel 45 according to various embodiments. The guide pin 30 can then be used to access the normal pedicle channel 45 to perform one or more procedures via the normal pedicle channel 45, e.g., insertion of a pedicle screw as fixation instrumentation.

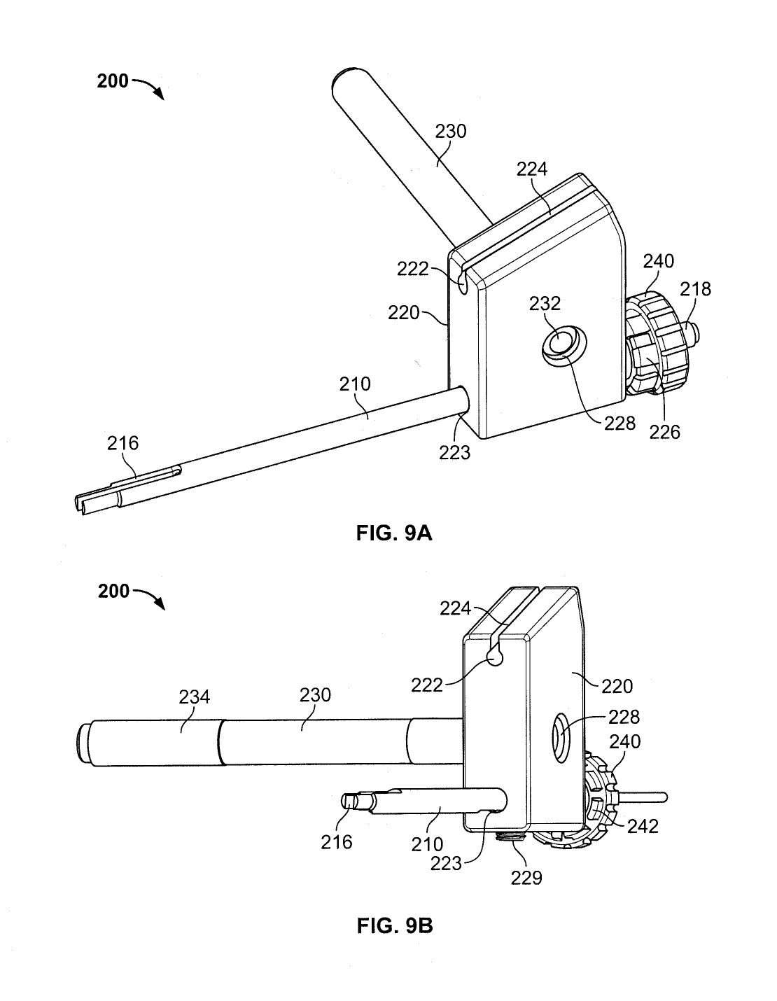

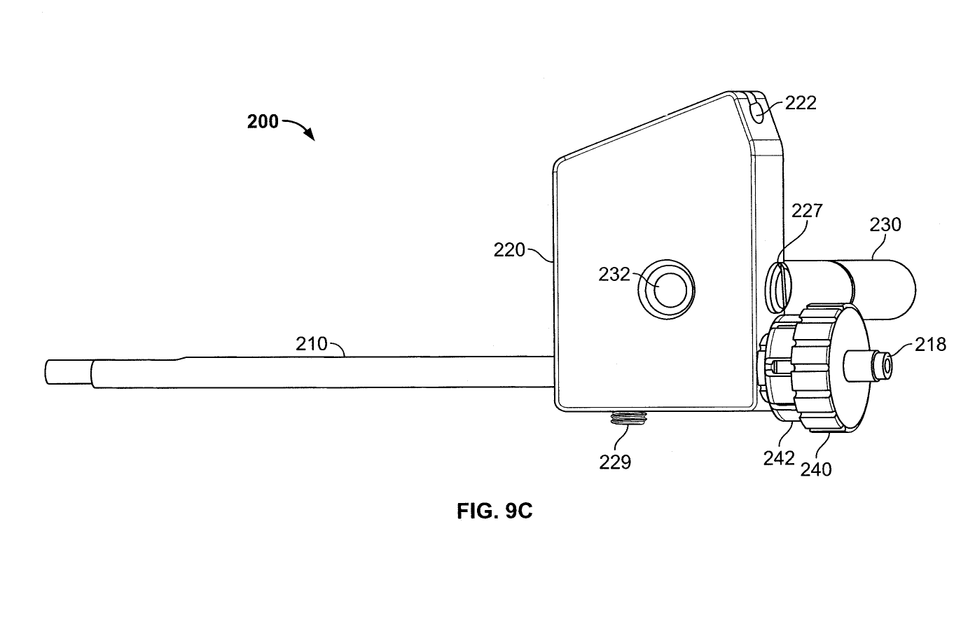

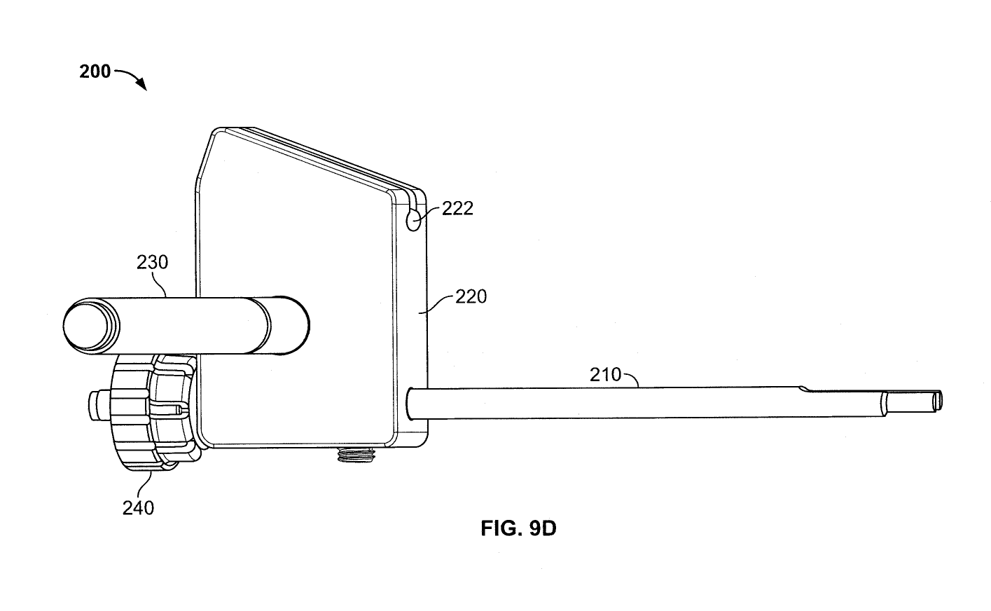

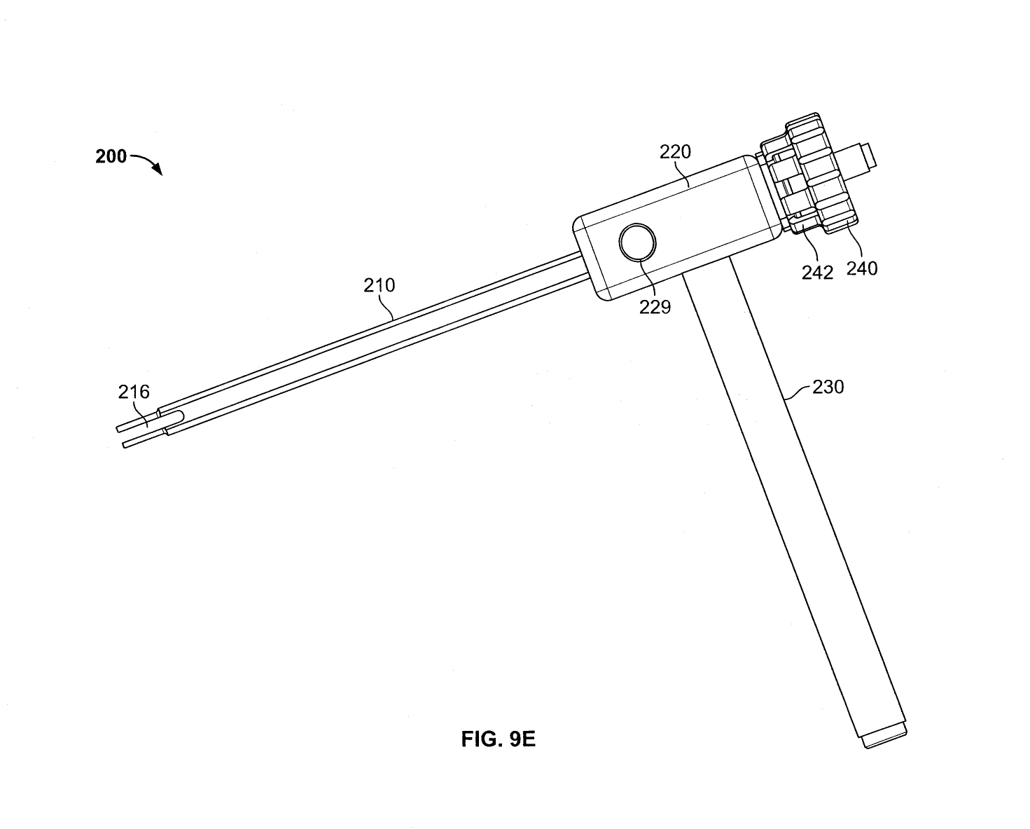

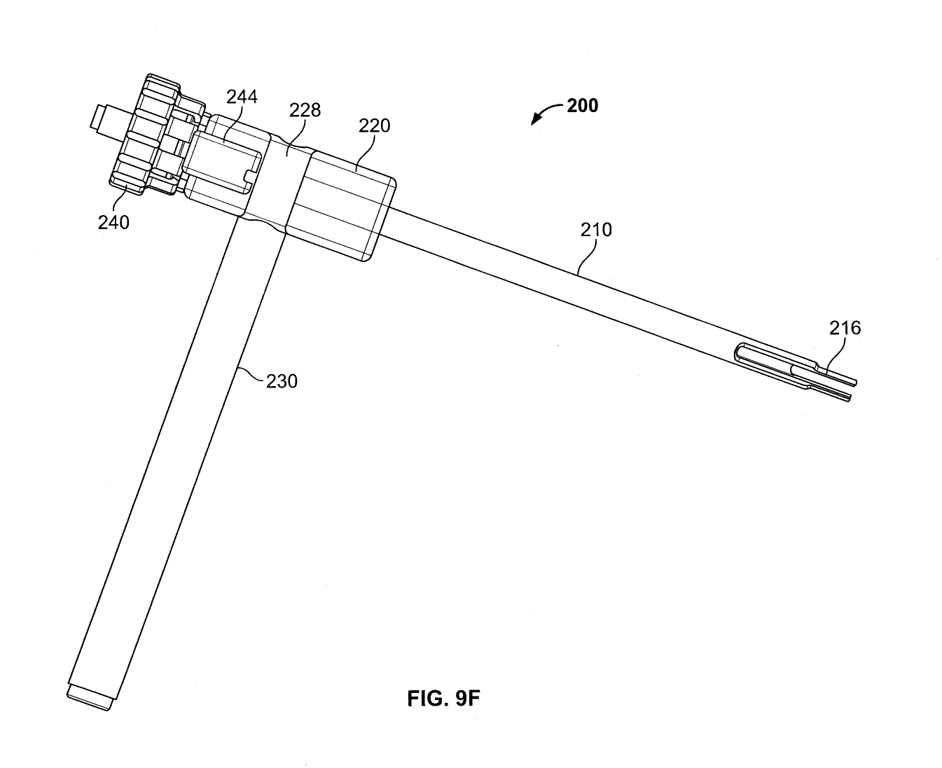

FIGS. 9A to 9F are diagrams of another transpedicular channel alignment and access tool system 200 according to various embodiments. As shown in these figures, the system 200 can include a length or extension adjustable, slotted 216, cannula 210, a cannula offset tool 220, a handle 230, and an extension or length adjustment knob 240 for the cannula 210. The handle 230 can transversely (relative to cannula 210) engage the offset tool 220 via a bore 228 and handle extension 232.

The offset tool 220 can include a first cannula channel 223 for cannula 210, a second channel 222 for a cannula or guide wire, a guide wire release slot 224, and a flange 228 for engaging one or more tabs 242 of the knob 240. As shown in FIG. 9B the handle 230 can include a larger, distal section 234. The channel 224 and cannula 210 slot 216 can be configured so a guide wire or other tool inserted into the channel 224 can pass through the cannula 210 via the slot 216. The system 200 can include a set pin or screw 229 in the offset tool 220 to fixably position the cannula 210 extension. As shown in FIG. 9C the system 200 can also include a set pin or screw 227 in the offset tool 220 to releasably engage the handle 230 so the handle extension 232 can be removed from the tool 220 channel 228. FIG. 9F includes a partial cross sectional view of the offset tool 220 showing a riser 244 that can be coupled to the knob 240 to enable translation of the cannula 210 slot 216.

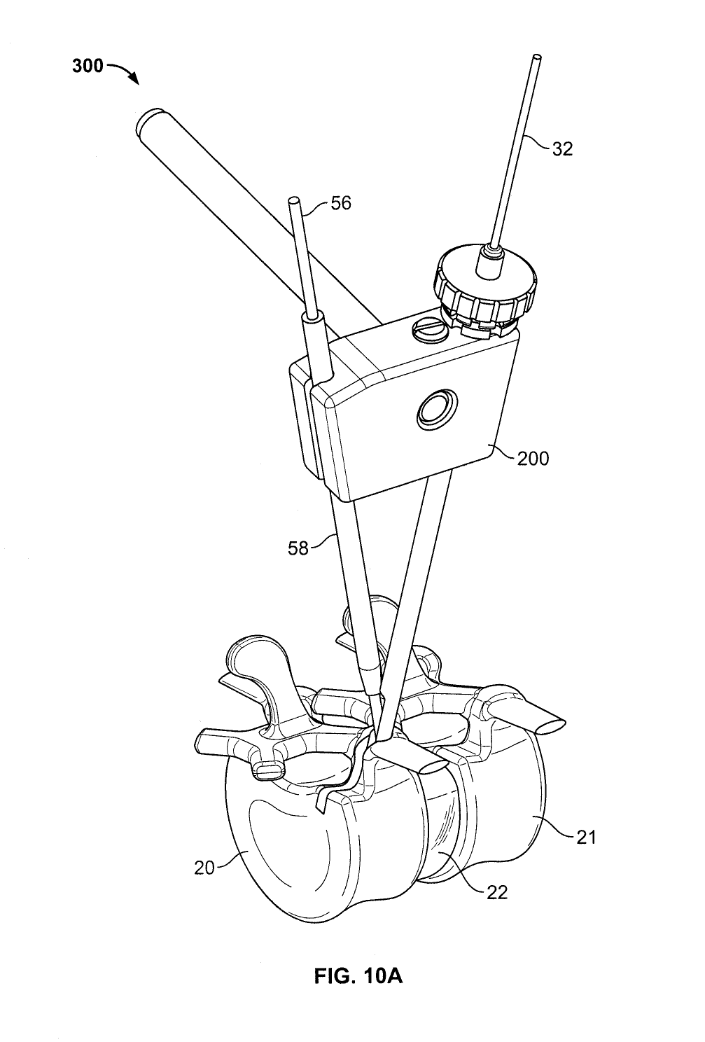

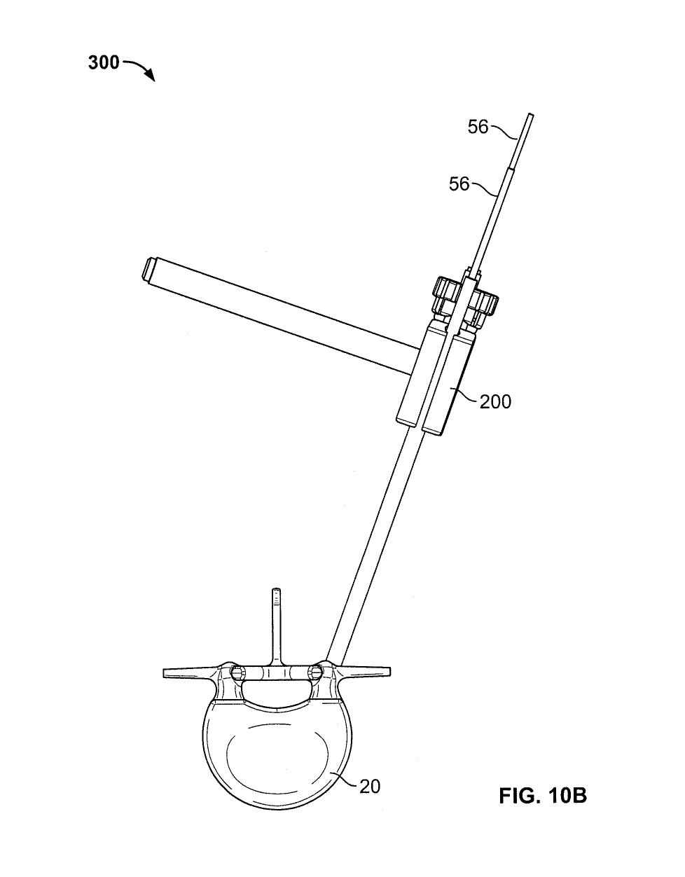

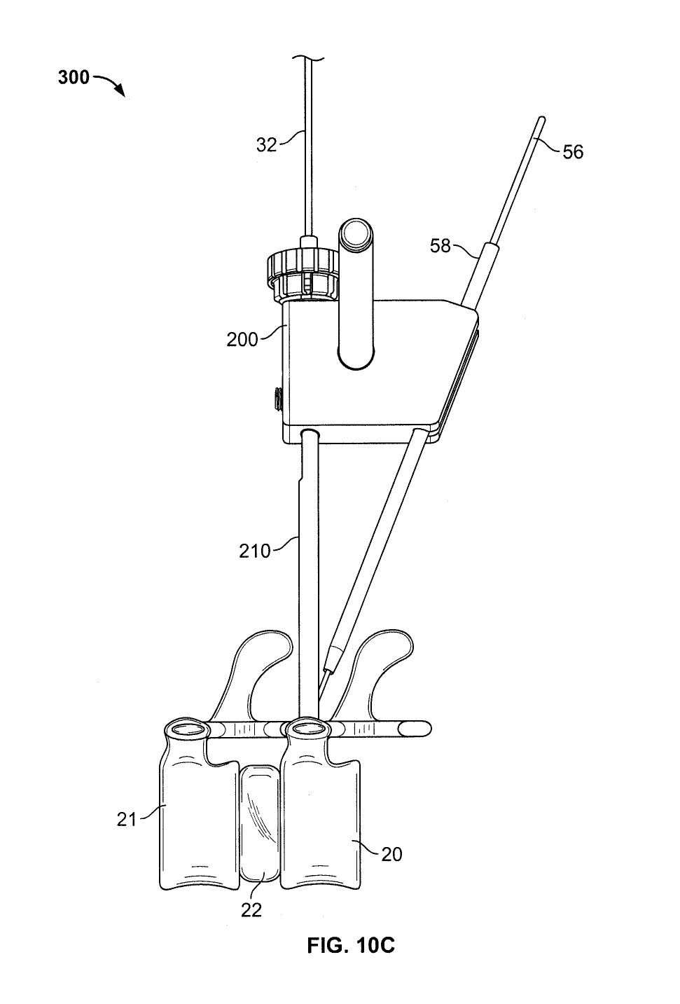

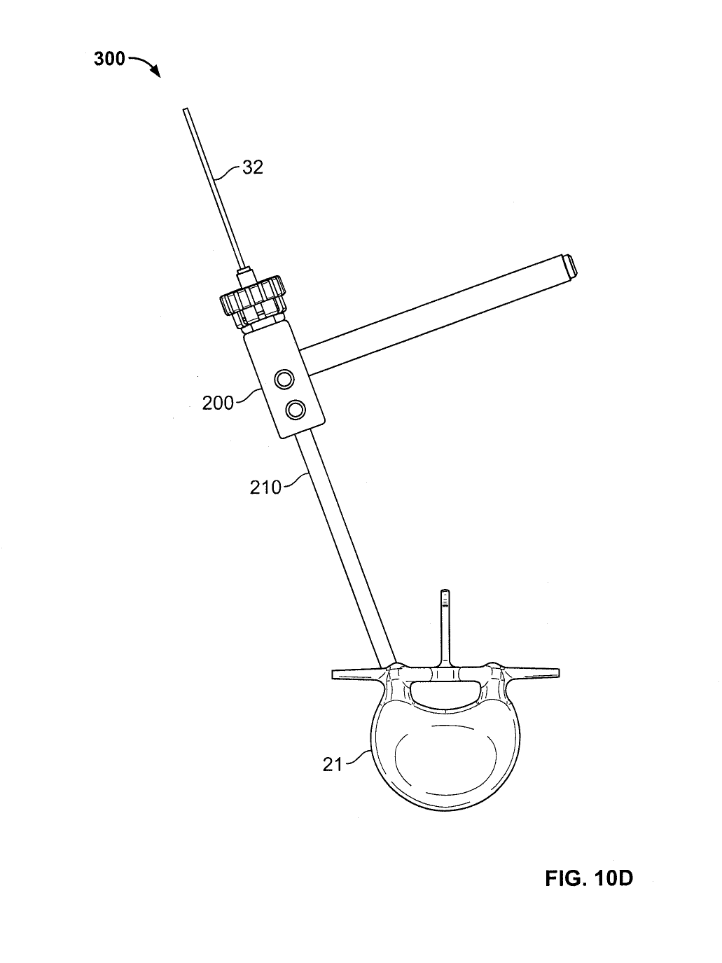

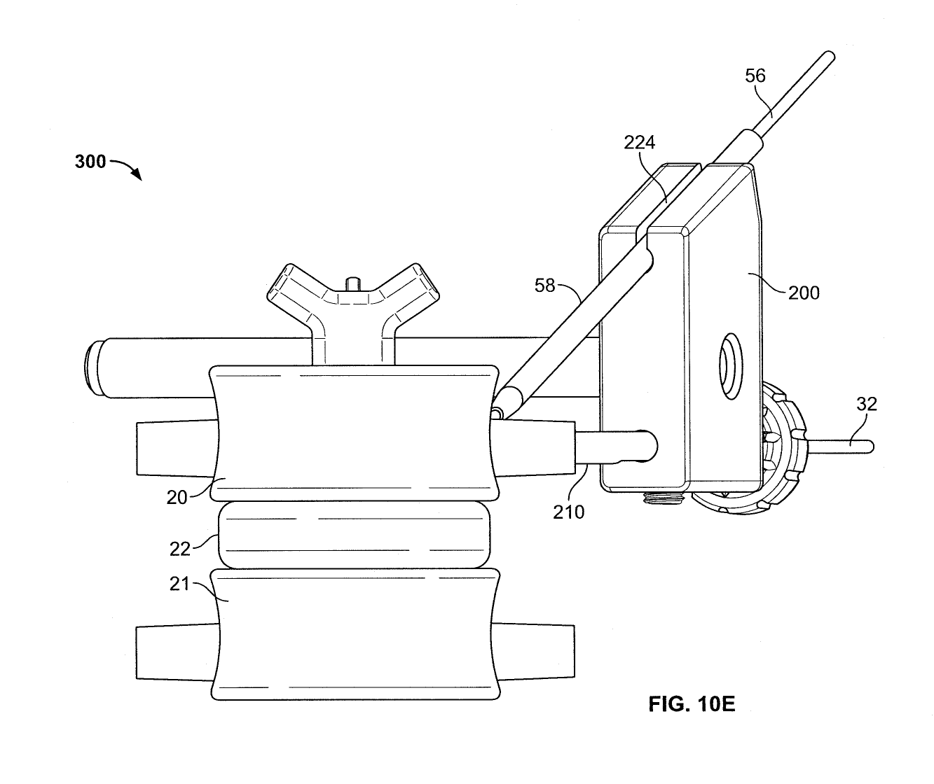

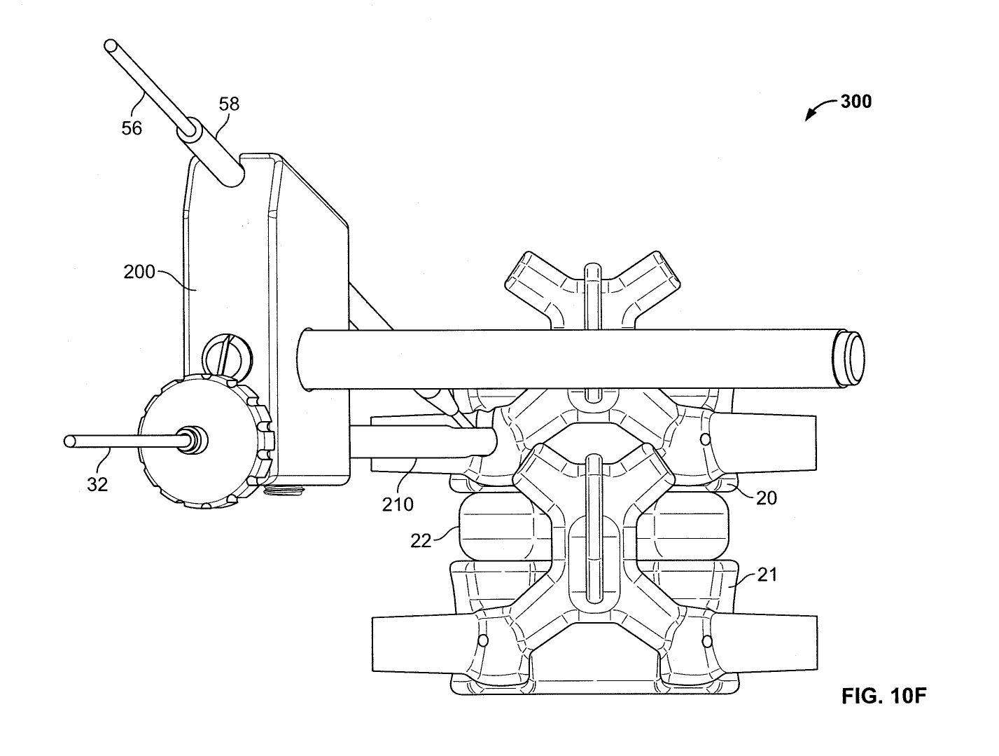

FIGS. 10A to 10F are diagrams of the transpedicular channel alignment and access tool system 300 employed in a vertebra 20 according to various embodiments. The cannula 210 can be inserted normally to the spinal vertebra 20 pedicle. A guide wire 56 and cannula 58 can be placed within the slot 222 of the tool 200. The guide wire 56 and cannula 58 can be inserted into the spinal vertebra 20, disc space 22, or adjacent spinal vertebra 21 as a function of the cannula 210 slot 216 translation via the knob 40. In an embodiment the cannula 210 can have a guide wire 32 inserted therein to securely engage the cannula 210 in the spinal vertebra 20. The guide wire 32 can be partially removed to enable guide wire 56 or cannula 58 to pass through the cannula 210 slot 216 and into one of the spinal vertebra 20, disc space 22, and adjacent spinal vertebra 21.

In an embodiment the knob 240 can be rotated to linearly translate the cannula 210. The cannula 210 translation can change the offset angle between the channel 222 and cannula 210. The offset between channel 222 and cannula 210 can enable a guide wire 58 or cannula 56 to engage the vertebra 20 when knob 240 is rotated to a first point. The offset between channel 222 and cannula 210 can enable a guide wire 58 or cannula 56 to engage the disc space 22 when knob 240 is rotated to a second point. The offset between channel 222 and cannula 210 can enable a guide wire 58 or cannula 56 to engage the adjacent vertebra 21 when knob 240 is rotated to a third point.

While this specification contains many specifics, these should not be construed as limitations on the scope of the claims or of what can be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A method of treating a spine comprising:
creating an intraosseous channel through a pedicle of a first vertebra, wherein the intraosseous channel extends along an axis of the pedicle from a generally posterior or posterior-inferior aspect to a generally anterior or anterior-superior aspect of the pedicle of the first vertebra;
accessing through at least a portion of the intraosseous channel a spinal region generally superior to the first vertebra and generally inferior to a second vertebra, wherein the spinal region comprises the neuroforamina, lateral recess, epidural space, or intervertebral disc space;
placing a treatment device through at least a portion of the channel into or adjacent the spinal region; and
performing a therapeutic intervention on the spinal region.

2. The method of claim 1, wherein the step of creating an intraosseous channel comprises using a first surgical guide.

3. The method of claim 2, wherein using a first surgical guide comprises inserting a portion of the first surgical guide within the intraosseous channel in the pedicle, the guide generally disposed along the posterior or posterior-inferior aspect of the axis of the pedicle.

4. The method of claim 3, wherein the step of accessing through at least a portion of the intraosseous channel the spinal region comprises using a second surgical guide, the second surgical guide having a first axis and a second axis, wherein the second axis is offset at a predetermined angle from the first axis.

5. The method of claim 4, wherein using a second surgical guide comprises extending the first axis of the second surgical guide generally along the posterior or posterior-inferior aspect of the pedicle and extending the second axis of the second surgical guide into or adjacent the spinal region generally superior to the first vertebra and generally inferior to the second vertebra.

6. The method of claim 5, wherein the portion of the channel accessing the spinal region is at least partially confluent with the posterior or posterior-inferior aspect of the intraosseous channel through the pedicle.

7. The method of claim 1, wherein the step of placing a treatment device comprises inserting an intervertebral distraction device through at least a portion of the channel.

8. The method of claim 7, wherein the step of performing a therapeutic intervention comprises distracting the first and second vertebrae with the intervertebral distraction device.

9. The method of claim 8, further comprising the step of placing a fixation device through at least a portion of the intraosseous channel in the posterior or posterior-inferior aspect generally along a posterior aspect of the axis of the pedicle.

10. The method of claim 9, wherein the fixation device comprises a pedicle screw.

11. The method of claim 1, wherein the step of performing a therapeutic intervention comprises delivering material into the spinal region or a space adjacent thereto.

12. The method of claim 11, wherein the material is selected from the group comprising bone growth materials, osteoconductive, osteoinductive, chondroproliferative, chondroreparative, growth factors, osteoproliferative materials, osteogenic proteins, osteoprogenic factor 1, bone morphogenetic proteins (BMP), BMP2 and BMP7.

13. The method of claim 1, further comprising the step of placing a fixation device through at least a portion of the intraosseous channel in the posterior or posterior-inferior aspect generally along a posterior aspect of the axis of the pedicle.

14. The method of claim 13, wherein the fixation device comprises a pedicle screw.

15. The method of claim 1, wherein the step of placing a treatment device comprises inserting a resection device through at least a portion of the channel into the spinal region or a space adjacent thereto.

16. The method of claim 15, wherein the step of performing a therapeutic intervention comprises resecting, shaving, shearing, cutting or removing disc material using the resection device.

17. The method of claim 16, further comprising the step of placing a fixation device through at least a portion of the intraosseous channel in the posterior or posterior-inferior aspect generally along a posterior aspect of the axis of the pedicle.

18. The method of claim 17, wherein the fixation device comprises a pedicle screw.

19. The method of claim 1, further comprising the step of delivering material into the spinal region or a space adjacent thereto.

20. The method of claim 19, wherein the material is selected from the group comprising bone growth materials, osteoconductive, osteoinductive, chondroproliferative, chondroreparative, growth factors, osteoproliferative materials, osteogenic proteins, osteoprogenic factor 1, bone morphogenetic proteins (BMP), BMP2 and BMP7.

21. A method of treating a spine comprising:
creating an intraosseous channel through a pedicle of a first vertebra, wherein the intraosseous channel extends along an axis of the pedicle from a generally posterior or posterior-inferior aspect to a generally anterior or anterior-superior aspect of the pedicle of the first vertebra, wherein the channel is created using a first surgical guide;
accessing through at least a portion of the intraosseous channel a spinal region generally superior to the first vertebra and generally inferior to a second vertebra, wherein the spinal region is accessed using a second surgical guide having a first axis and a second axis, wherein the second axis is offset at a predetermined angle from the first axis;
placing an intervertebral distraction device through at least a portion of the channel into or adjacent the spinal region;
distracting the first and second vertebrae with the intervertebral distraction device;
inserting a resection device through at least a portion of the channel into the distracted spinal region or a space adjacent thereto and resecting, shaving, shearing, cutting or removing disc material using the resection device; and
placing a fixation device through at least a portion of the intraosseous channel in the posterior or posterior-inferior aspect generally along a posterior aspect of the axis of the pedicle.

22. The method of claim 21, wherein the spinal region comprises the neuroforamina, lateral recess, epidural space, or intervertebral disc space.

23. The method of claim 21, further comprising the step of delivering material into the spinal region or a space adjacent thereto.

24. The method of claim 23, wherein the material is selected from the group comprising bone growth materials, osteoconductive, osteoinductive, chondroproliferative, chondroreparative, growth factors, osteoproliferative materials, osteogenic proteins, osteoprogenic factor 1, bone morphogenetic proteins (BMP), BMP2 and BMP7.
